(19) United States
(12) Patent Application Publication
(10) Pub. No.: US 2026/0256513 A1
(43) Pub. Date: Sep. 3, 2026

(54) DEVICES FOR VESSEL DEBULKING WITH RADIOFREQUENCY ABLATION

(71) Applicant: Alaris Vascular, Inc., Solana Beach, CA (US)

(72) Inventors: Christopher Jordan Hasselwander, Carlsbad, CA (US); Steven Charles Howard, La Jolla, CA (US); Brad Klos, Solana Beach, CA (US)

(73) Assignee: Alaris Vascular, Inc., Solana Beach, CA (US)

(21) Appl. No.: 19/644,280

(22) Filed: Apr. 10, 2026

Related U.S. Application Data
(63) Continuation of application No. PCT/US2025/060097, filed on Dec. 17, 2025.
(60) Provisional application No. 63/735,499, filed on Dec. 18, 2024.

(51) Int. Cl. A61B 18/14; A61B 17/00; A61B 17/12; A61B 18/00; A61B 90/00; A61F 2/82; A61M 25/09; A61M 25/10

(52) U.S. Cl. CPC A61B 18/1492; A61F 2/82; A61B 2017/00867; A61B 17/12136; A61B 2018/0016; A61B 2018/0022; A61B 2018/0041; A61B 2018/00791; A61B 2018/0091; A61B 2018/1467; A61B 2090/3966; A61M 25/09; A61M 2025/1061

(57) ABSTRACT

A device for treating a target lesion may include an elongate body comprising a tissue modification device on a distal end of the elongate body. The tissue modification device may include an electrode array and an expandable support structure configured to support the electrode array. When expanded, the expandable support structure is configured to space adjacent electrodes of the electrode array at a substantially equal distance. Further, when expanded, the electrode array is configured to generate one or more tissue modification energy fields around the expandable support structure when an electrical current is supplied to the electrode array. A method of treating a target lesion may include advancing a tissue modification device to a target tissue; expanding the tissue modification device to expand an electrode array; and generating a plurality of zones of radiofrequency (RF) fields through at least a part of the electrode array to ablate the target lesion.

### CROSS-REFERENCE TO RELATED APPLICATIONS

This application is a continuation of International Patent Application Ser. No. PCT/US2025/060097, filed Dec. 17, 2025, which claims the priority benefit of U.S. Provisional Patent Application Ser. No. 63/735,499, filed Dec. 18, 2024, the contents of each of which is herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of vessel or implantable device debulking and, more specifically, to the field of vessel or implantable device debulking utilizing energy fields. Described herein are systems and methods for debulking of target tissues by densifying lesions in the target tissue.

### BACKGROUND

Post-thrombotic syndrome (PTS) arising from chronic venous outflow obstruction represents one of the most costly and morbid sequelae of chronic venous insufficiency. Conservative therapies are largely palliative, and definitive management often requires endovenous stenting. However, patients with PTS experience disproportionately high rates of in-stent restenosis (ISR), reported at 40-70% within 12 months, due to organized thrombus, fibrosis, and dense collagen deposition. ISR in this population is associated with recurrent pain, edema, ulceration, and progressive venous hypertension, creating a substantial unmet clinical and economic burden. Current reinterventions, including balloon angioplasty or hyperdilation, provide only transient luminal gain. More aggressive strategies can include mechanical atherectomy with embolic protection or surgical bypass.

### SUMMARY

In some aspects, the techniques described herein relate to a tissue modification device for modification of a target tissue within a vessel, the tissue modification device including: an elongate body including the tissue modification device on a distal end of the elongate body, the tissue modification device including: an electrode array, and an expandable support structure configured to support the electrode array, wherein, when expanded, the expandable support structure is configured to space adjacent electrodes of the electrode array at a substantially equal distance, and wherein the electrode array is configured to generate one or more tissue modification energy fields around the expandable support structure when an electrical current is supplied to the electrode array.

In some aspects, the techniques described herein relate to a system for debulking of a target tissue from a reusable tissue modification device in a vessel, the system including: an elongate body including a tissue modification device on a distal end of the elongate body, the tissue modification device including: a scaffold including an electrode array with about evenly spaced adjacent electrodes; an expandable support structure coupled to the scaffold and configured to expand the scaffold; and a radiofrequency (RF) generator configured to supply an electrical current to the electrode array and generate a RF field for modification of the target tissue.

In some aspects, the techniques described herein relate to a method of treating a target lesion, the method including: advancing a tissue modification device to a target tissue including the target lesion, wherein the tissue modification device includes: an expandable support structure, and an electrode array at least partially surrounding the expandable support structure, wherein, when the expandable support structure is expanded, the electrode array including adjacent electrodes that are evenly spaced; expanding the expandable support structure to expand the electrode array; and generating a plurality of zones of radiofrequency (RF) fields through at least a part of the electrode array to activate the electrode array to ablate the target tissue.

In some aspects, the techniques described herein relate to the method of 69, wherein the contracting, re-positioning, and re-deploying occur more than one time.

In some aspects, the techniques described herein relate to a system for treating restenosis of an implantable device, including: an elongate body including an expandable support structure at least partially surrounded by an electrode array on a distal end of the elongate body, wherein the electrode array is configured for directional ablation, and wherein the expandable support structure is configured to expand the electrode array against a lesion associated with the implantable device; and a control system including a radiofrequency (RF) field generator configured to selectively activate angular zones of the electrode array, wherein the control system is configured to perform a feedback loop to maintain an ablation temperature within a therapeutic range.

In some aspects, the techniques described herein relate to a system for debulking of a target tissue from a reusable tissue modification device in a vessel, the system including: an elongate body including a tissue modification device on a distal end of the elongate body, the tissue modification device including: an electrode array coupled to a scaffold, wherein: the scaffold configured to expand and contract, and when expanded, the scaffold is configured to space adjacent electrodes of the electrode array at a substantially equal distance; and a radiofrequency (RF) field generator configured to supply an electrical current to the electrode array and generate an RF field for modification of the target tissue.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates a perspective view of an embodiment of a tissue modification device for tissue manipulation at a target tissue.

FIG. 2 illustrates a perspective view of an embodiment of a system for debulking of a target tissue.

FIG. 3 illustrates a box diagram of an embodiment of a system for debulking a target tissue.

FIG. 4A illustrates a side view of an embodiment of a distal end of a system with an outer sheath axially advanced to cover the tissue modification device.

FIG. 4B illustrates a side view of the embodiment of the distal end of the system of FIG. 4A with the outer sheath retracted to expose the expandable support structure and electrode array.

FIG. 4C illustrates a side view of the embodiment of the distal end of the system of FIG. 4A with the expandable support structure expanded to support the electrode array.

FIG. 4D shows a zoomed-in view of an electrode of the embodiment of FIG. 4C.

FIG. 4E shows a zoomed-in view of an orientation indicator of the embodiment of FIG. 4C.

FIG. 5 illustrates a perspective side view of an embodiment with an expandable support structure that includes a scaffold.

FIG. 6 illustrates a perspective side view of an embodiment with the electrode array with an expandable support structure that includes a scaffold and a balloon.

FIG. 7A illustrates a perspective view of an embodiment of a system for debulking a target tissue with an expandable support structure that includes the electrode array printed on the balloon.

FIG. 7B illustrates a cross-sectional view of an embodiment of a distal end of a tissue modification device showing the conductors.

FIG. 8A illustrates a perspective view of an embodiment of a distal end of a system with an expandable support structure expanded to support an electrode array that is generating a plurality of energy fields.

FIG. 8B illustrates a transverse cross-sectional view of an embodiment of the distal end of the system of FIG. 8A.

FIG. 9A illustrates a channel map of a cross-section of a tissue modification device of an embodiment showing the related energy field channels utilized to generate an energy field.

FIG. 9B illustrates a linear channel map of an embodiment with positive and negative electrodes generating an energy field for target tissue ablation.

FIGS. 10A-10B illustrate perspective views of an embodiment of a handle for a tissue debulking system.

FIGS. 11A-11B illustrate a side and cross-sectional view, respectively, of an embodiment of a handle with one or more channels for electrical connections, guide mechanisms, and expandable support structure controls.

FIGS. 12A-12F illustrate top views of various embodiments of electrode arrays that may be utilized to generate energy field ablation for a target tissue.

FIGS. 13A-13D illustrate perspective views of various embodiments of electrode arrays that may be utilized to generate energy field ablation for a target tissue.

FIGS. 14A-14C illustrate a perspective view, side view, and end view, respectively, of an embodiment of a scaffold of a distal end of a system.

FIG. 15 illustrates a flow chart of an embodiment of a restenosis treatment method for debulking a target tissue.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Conventional therapies such as high-pressure balloons, cutting balloons, or atherectomy are constrained by elastic recoil, vessel injury, and inconsistent outcomes. Repeat stenting, drug-coated balloons, and prolonged anticoagulation have also failed to demonstrate success in the venous system. Further, balloon angioplasty or hyperdilation, provide only transient luminal gain. More aggressive strategies, such as mechanical atherectomy with embolic protection, add procedural complexity and risk, while surgical bypass remains an invasive last resort with limited durability.

To solve the above technical problems, the systems described herein may include expansion of an expandable support structure with controlled radiofrequency energy delivery. For example, a tissue debulking device may be advanced over a guiding device (e.g., guidewire) and positioned across a restenotic implantable device. The expandable support structure may be inflated to nominal pressure or expanded to establish tissue contact with a scaffold of the device. During inflation, energy may be delivered through electrodes embedded in the scaffold, generating a bipolar or monopolar field that may be tailored to local tissue impedance, at least in some embodiments. The energy may be deposited into fibrotic ISR tissue while sparing the scaffold, since the impedance differential between neointimal tissue and the material of the scaffold prevents energy coupling to the scaffold. This characteristic may be particularly effective for eccentric ISR lesions, as it allows targeted ablation of tissue without collateral device heating. Additionally, expandable support structure reinforcement with the scaffold enhances fatigue resistance, facilitates reliable deflation, and enables faster repositioning. This durability allows a single device to treat long, diffuse ISR zones typical of iliofemoral disease, thereby improving efficiency and reducing the need for multiple catheters.

In some embodiments, the system may include a catheter-based energy field ablation device for treatment of ISR in vascular applications. The system may include an energy field generator with optional user interface, an interface cable, and an over-the-wire catheter incorporating an expandable support structure-actuated electrode array which may be printed on the expandable support structure or mounted to a scaffold to ensure reliable, repeated deployment. In some embodiments, the device may include an integrated hydrophilic coated outer sheath, which may be used to recapture the expandable support structure-electrode array. The elongate body may have sufficient flexural rigidity and torsional response to ensure delivery to the affected patient area and terminates with an atraumatic tip. In some embodiments, upon deployment, the expandable support structure inflates up to about 4 atm; up to about 6 atm; up to about 8 atm; up to about 12 atm; up to at least about 10 atm; or to between about 8 atm and about 15 atm to compact the lesion and direct one or more independently controllable zones of energy fields out toward the lesions for modification. In some embodiments, the expandable support structure is inflated with a fluid (e.g., saline, gas, contrast, etc.). In some embodiments, upon deployment, the expandable support structure inflates to at least about 4 atm; to at least about 6 atm; to at least about 8 atm; to at least about 12 atm; to at least about 10 atm; or to between about 4 atm and about 15 atm to compact the lesion.

The design of the tissue modification devices described herein permits repeated deployment without structural fatigue, while radiopaque markers aid visualization throughout the workflow.

In some embodiments, the systems, devices, and methods described herein provide a further technical solution of using radiofrequency energy delivery with balloon angioplasty to cause thermal remodeling of tissue within restenotic implantable devices (e.g., stents), controlled implantable device debulking, and/or luminal expansion. Said another way, the systems, devices, and methods described herein use peripheral radiofrequency-mediated thermal ablation with active compression (e.g., balloon expansion) to achieve durable debulking of ISR to modify the tissues through densification, resulting in fewer reinterventions. In some embodiments, the systems described herein may use controlled thermal denaturation of collagen and fibrin within neointimal tissue to reduce lesion burden and improve long-term patency. Further, the systems described herein may avoid overheating, coagulum formation, and/or inefficient energy transfer to promote consistent ablation performance across varying lesion morphologies. In some embodiments, the systems described herein may selectively debulk implantable devices by adjusting a depth of the lesion that is treated and/or by selectively activating one or more electrodes or electrode zones to generate an energy field in that zone, for example to target a particular lesion or target tissue. The zones may treat particular lesions in the target tissue site(s) and provide different energy fields for treatment should a higher temperature or deeper treatment be needed. Dilatation due to radial pressure of the expandable support structure(s) may compress lesions before densifying; otherwise, densification without compression may result in the vessel or implantable device remaining occluded.

Additionally, or alternatively, the systems and devices described herein may provide the further technical solution of evenly spaced or substantially equally spaced electrodes that may be used to debulk the target tissue. The systems and devices described herein may include a plurality of electrodes disposed at a substantially equal distance from one another when the expandable support structure is in an expanded state. The substantially equal spacing between adjacent electrodes may be between about 2 mm and about 6 mm, for example about 4 mm. The spacing is selected to achieve a predictable depth of radiofrequency (RF) energy penetration into the target tissue. For example, a spacing of about 4 mm may correspond to a bipolar RF field penetration depth of about 1 mm to about 9 mm, for example about 4 mm, thereby enabling controlled thermal remodeling of restenotic tissue without excessive collateral damage. Maintaining consistent electrode spacing may enable a uniform energy field distribution and clinical efficacy, as irregular spacing may result in uneven ablation, incomplete lesion debulking, and/or localized overheating. In some embodiments, the number of electrodes may be determined based on a mathematical relationship between the circumference of the expanded balloon and the target electrode spacing, such that the electrode array provides circumferential coverage while preserving the desired spacing to optimize lesion densification and restore luminal patency.

A further technical advantage is that, at least in some embodiments, the tissue modification device may allow for multiple recapture and redeployment cycles within a single procedure. The expandable support structure (e.g., balloon and/or scaffold) may be coupled to a cuff designed to withstand repeated axial translation without structural fatigue. Materials such as Nitinol for the scaffold and Polyether Ether Ketone (PEEK) for the cuff may be utilized to provide durability and facilitate repeated repositioning.

The systems and devices described herein may utilize a tissue modification device that may be expanded for use and contracted (e.g., into the unexpanded state) and removed for reuse or after procedure completion. The tissue modification devices may include an electrode array for generating a tissue modification energy field when an electric current is carried, and an expandable support structure (e.g., balloon and/or scaffold) for expanding the electrode array for use and then contracted for removal and reuse. In some embodiments, a tissue modification energy field may include a radiofrequency (RF) field, a pulsed field ablation, a microwave, or other ablating energy field. Other portions of the system may include an energy field generator for supplying the energy for generating tissue modification energy fields such as RF field(s), pulsed field ablation, or other energy fields that may be generated to densify tissues by the tissue modification device. In some embodiments, a system may include an elongate body with an outer sheath, axially movable over the elongate body, for guiding the tissue modification device through vasculature in the body to the target tissue and/or vessel. The elongate body may further organize one or more interface cables, ports, lumens, conductive materials, etc. to prevent entanglement and control placement. In some embodiments, a system may include a handle for controlling an orientation of the system, and use of the tissue modification device. For example, the handle may control energy delivery, fluid delivery (e.g., to expand an expandable member), power delivery, and the like.

In some embodiments, the system may include orientation indicia disposed on one or more components (e.g., scaffold, expandable support structure, distal tip, etc.) of the tissue modification device and/or handle. The indicia may include one or more radiopaque markers that can provide rotational orientation feedback under fluoroscopy. For example, the radiopaque markers may include asymmetric patterns, stripes, or arrows positioned on the outer sheath, expandable support structure, and/or scaffold such that the angular position of one or more electrode or electrode zones is visually discernible. In some embodiments, the handle may include corresponding indicia aligned with the radiopaque markers to provide tactile or visual feedback regarding the rotational orientation of the electrode array relative to the target tissue.

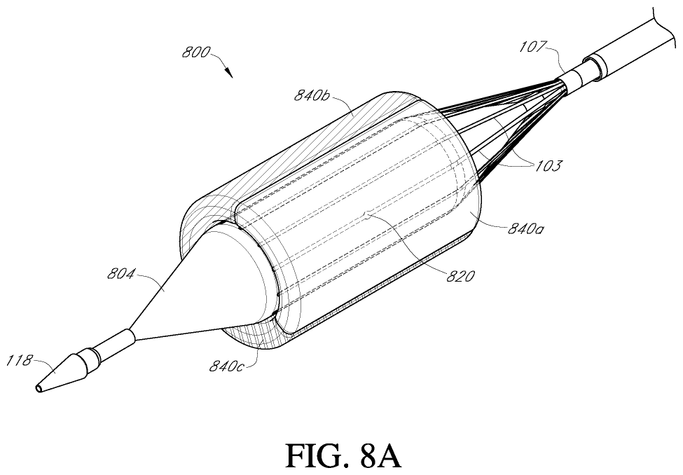

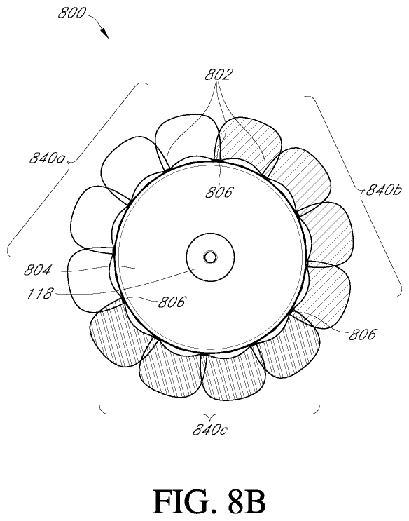

In some embodiments, the devices described herein may include an electrode configuration, one or more electrode configurations, or a plurality of electrode configurations in an electrode array and/or one or more electrode zones. The one or more electrode zones may be selectable or combinable for targeted directional treatment. At least a portion of the one or more electrode zones may include or be formed of radiopaque material (e.g., gold, platinum, tantalum, titanium, stainless steel, a combination thereof, etc.) such that the one or more electrodes and/or electrode zones are identifiable using fluoroscopy. In some embodiments, the electrode array may include one or more electrode zones based on electrode structure and operating mode (i.e., monopolar or bipolar). Each electrode zone may be operated in monopolar or bipolar energy field mode (e.g., based on one or more inputs, for example, into a display of the system or otherwise received by the system) to provide energy field(s) to a target lesion depth. In some embodiments, the operating mode of the electrodes may be switched between bipolar and monopolar electrodes. In some embodiments, the electrode zones may include angular zones, linear zones, and/or a combination thereof. In some embodiments, the angular zones include a range of evenly spaced zones as shown, for example, in FIGS. 8A-8B. In FIGS. 8A-8B, three angular zones 140a, 140b, 140c cover about 120 degrees each to cover the circumference of the tissue modification device 101. In this manner, if the target lesion(s) are eccentric and thus may be treated using an angular zone of the tissue modification device 101, then only the angular zone(s) in contact with, or in proximity to, the lesion(s) needs to be activated. In some embodiments, more than three angular zones may be utilized. Each zone may evenly cover the circumference of the tissue modification device 101. For example, four angular zones may include zones covering about 90 degrees, five angular zones may include zones covering about 74 degrees, and six angular zones may include zones covering about 60 degrees. Alternatively, each zone may have a unique circumferential distribution, such that a first zone covers a first set of circumferential degrees, a second zone covers a second set of circumferential degrees, and so forth.

In some embodiments, the zones may include axial zones that run linearly. In such an embodiment, each electrode may instead be separated along the axial length of the tissue modification device 101 and therefore the distal end of the device may be activated separate from the proximal end. In some embodiments, more even linear spacing may be utilized; however, similar to the angular zones, the linear zones may also evenly spaced along the about 20 mm to about 40 mm length of the tissue modification device 101. In some embodiments, both linear and angular zones may be utilized. For example, six zones may cover the length and circumference of the tissue modification device 101. In this example, each zone may cover about 120 degrees of each side of the circumference and about 20 mm of the proximal or distal length of the tissue modification device 101.

In some embodiments, the systems described herein may employ bipolar electrodes. In some embodiments, the systems described herein may employ monopolar electrodes. In some embodiments, the systems described herein may employ bipolar and monopolar electrodes such that the system may be operated in a bipolar mode or monopolar mode. In some embodiments, lesion depth may be mode-dependent, with safety cutoffs for impedance changes incorporated into the generator software to prevent overheating or unintended implantable device contact during use. In some embodiments, a bipolar mode may achieve depths of about 2 mm to about 6 mm. For example, bipolar mode may penetrate lesions up to a depth of between about 0.1 mm to about 6 mm; about 0.5 mm to about 5 mm; about 1 mm to about 4 mm; about 2 mm to about 4 mm; about 3 mm to about 5 mm; about 3.5 mm to about 4.5 mm; etc. In some embodiments, monopolar mode may achieve depths of about 5 mm to about 10 mm. For example, monopolar mode may penetrate lesions up to a depth of between about 0.1 mm to about 12 mm; about 1 mm to about 10 mm; about 2 mm to about 9 mm; about 2 mm to about 8 mm; about 4 mm to about 9 mm; about 5 mm to about 9 mm; about 6 mm to about 9 mm; about 7 mm to about 9 mm; about 7.5 mm to about 8.5 mm; etc. In some embodiments, different or additional sensors for impedance and power output may be utilized as well for real-time feedback looping to determine whether the ranges of temperature, impedance, and/or power output are within a therapeutic range.

In some embodiments, the tissue modification device may include one or more electrodes printed directly on the expandable support structure, such that the expandable support structure provides both occlusive pressure and electrode support. In some embodiments, the one or more electrodes may be mounted on an independent scaffold layer decoupled from the expandable support structure, such that the scaffold maintains consistent electrode spacing during expandable support structure inflation and deflation. In some embodiments, a tissue modification device may include a vector-style device comprising a radially expandable scaffold formed of a shape-memory material (e.g., Nitinol) without a balloon, such that the radially expandable scaffold applies outward radial force to press one or more electrodes against a vessel wall. Each embodiment may be configured to maintain substantially equal electrode spacing and/or enable selective activation of angular or linear zones of one or more electrodes.

In some embodiments, an outer diameter of the tissue modification device (e.g., in the expanded state) may be between about 8 mm to about 16 mm based on the diameter of the implantable device and/or vein (i.e., site of the tissues to be treated). In some embodiments, the outer diameter (i.e., the circumference) of the tissue modification device may determine the number of electrodes in the array and/or number of energy fields, e.g., RF fields, generated based on about even distribution of the electrodes. For example, when in an expanded state, the electrodes of an about 10 mm outer diameter device may include electrodes that are separated by about 2 mm to about 6 mm, but for an about 8 mm outer diameter device, separation may be about 3 mm to about 5 mm. The separation between the electrodes may result in a penetration of the energy fields by about 4 mm, in some embodiments. To accommodate variations in patient anatomy and lesion severity, the electrode spacing may be optimized for the diameter of each expandable support structure.

In some embodiments, the electrode array may be expanded and contracted a plurality of times to ablate a plurality of portions of a target tissue or an entire length of a target tissue, for example, in an implantable device. In some embodiments, the electrode array may be expanded and contracted as needed, re-ablating if a previous treatment did not work as effectively.

The described systems, devices, and methods may be utilized to treat thrombus or thrombi in the iliofemoral vein, femoral vein, iliac vein, deep vein thrombosis, and/or other vascular stent thrombosis. The described systems, devices, and methods may be used to lesions in one or more vessels. Regardless of the indication, the systems and methods described herein may enable selective activation of less than 360 degrees of the electrode array to treat eccentric lesions while sparing healthy tissue. In some embodiments, the electrode array comprises a plurality of angular zones (e.g., three zones each covering about 120 degrees) or linear zones along the axial length of the device. Selective activation of one or more zones may be based on preoperative imaging (e.g., intravascular ultrasound, a computed tomography scan, etc.) or intraoperative fluoroscopic feedback. As described elsewhere herein, the systems, devices, and methods described herein may be used to treat lesions at a plurality of depths, for example, based on type of electrode (monopolar or bipolar), a number of activated zones, an energy applied, etc.

The described systems, devices, and methods may be utilized to treat ISR in the iliofemoral vein, femoral vein, iliac vein, deep vein thrombosis, and/or other vascular stent thrombosis. The described systems, devices, and methods may be used to treat Post-Thrombotic-Syndrome related ISR. Regardless of the indication, the systems and methods described herein may enable selective activation of less than 360 degrees of the electrode array to treat eccentric lesions while sparing healthy tissue. In some embodiments, the electrode array comprises a plurality of angular zones (e.g., three zones each covering about 120 degrees) or linear zones along the axial length of the device. Selective activation of one or more zones may be based on preoperative imaging (e.g., intravascular ultrasound, a computed tomography scan, etc.) or intraoperative fluoroscopic feedback.

In some embodiments, the systems described herein may reduce lesion depth by about 0.5 mm to about 5.0 mm; about 1 mm to about 5 mm; about 1.5 mm to about 4 mm; about 2 mm to about 4 mm; about 3 mm to about 4 mm; etc. The therapy may be delivered for about 5 seconds to about 5 minutes; about 10 seconds to about 5 minutes; about 30 seconds to about 5 minutes; about 30 seconds to about 60 seconds; about 45 seconds to about 3 minutes; about 45 seconds to 75 seconds; about 60 seconds to about 90 seconds; about 30 seconds to about 200 seconds; etc. The thermal ablation may reach between about 40 degrees Celsius (i.e., about 104 degrees Fahrenheit) to about 37.8 degrees Celsius (i.e., about 100 degrees Fahrenheit); about 40 degrees Celsius (i.e., about 104 degrees Fahrenheit) to about 90 degrees Celsius (i.e., about 194 degrees Fahrenheit); about 70 degrees Celsius (i.e., about 158 degrees Fahrenheit) to about 85 degrees Celsius (i.e., about 185 degrees Fahrenheit); about 75 degrees Celsius (i.e., about 167 degrees Fahrenheit) to about 85 degrees Celsius (i.e., about 185 degrees Fahrenheit); etc. The tissue interface temperature may be between about 40 degrees Celsius (i.e., about 104 degrees Fahrenheit) to about 100 degrees Celsius (i.e., about 212 degrees Fahrenheit); about 40 degrees Celsius (i.e., about 104 degrees Fahrenheit) to about 90 degrees Celsius (i.e., about 194 degrees Fahrenheit); about 70 degrees Celsius (i.e., about 158 degrees Fahrenheit) to about 85 degrees Celsius (i.e., about 185 degrees Fahrenheit); about 75 degrees Celsius (i.e., about 167 degrees Fahrenheit) to about 85 degrees Celsius (i.e., about 185 degrees Fahrenheit); etc.

In some embodiments, any of the tissue modification devices described herein may be controllable by a handle. For example, the components of the system, such as electrodes, guidewire, outer sheath, expandable support structure (e.g., balloon, scaffold, etc.), etc. may be controlled and/or maintained through the handle, preventing entanglement of these components. The handle may provide channels for connections to each component to prevent entanglement and provide the ability to control the components during utilization. The handle provides a mechanism to determine orientation and adjust orientation of the tissue modification device.

The handle may include orientation indicia corresponding to one or more radiopaque markers on a distal end of the device. The handle may further include a user interface for enabling zone selection, where one or more angular or linear zones of the electrode array may be selectively activated. The user interface may be implemented using one or more mechanical switches, software controls, or a combination thereof.

One or more radiopaque marker configurations on the tissue debulking device may allow visualization of an orientation of the electrode array and/or one or more zones so that the user may position the array in the most optimal orientation for treatment of a target lesion, target tissue, or eccentric lesion associated with an implantable device and/or associated with a vessel. The radiopaque marker configuration on the tissue debulking device may correspond to the one or more indicia on the handle.

In some embodiments, real-time temperature sensor (e.g., thermocouple) feedback may help maintain target tissue temperatures at about 70 degrees Celsius to about 90 degrees Celsius under generator power control. For example, in some embodiments, the system may include a temperature sensor disposed in each electrode zone to provide real-time temperature feedback. Each temperature sensor may be electrically coupled to the control system using independent conductors routed through the elongate body. The temperature sensors may be positioned substantially at a center of each zone to accurately monitor tissue temperature during ablation, although other locations within each zone are also contemplated herein. Alternatively, or additionally, in some embodiments, one or more temperature sensors may be disposed on a scaffold, at one or more locations on an expandable support structure, on a tip of the system, or otherwise. The location of one or more temperature sensors may be correlated to a location of one or more electrodes or may be independent of electrode placement. In some embodiments, the system may maintain tissue temperature within a therapeutic range of about 40 degrees Celsius to about 95 degrees Celsius or about 60 degrees Celsius to about 95 degrees Celsius, for example about 80 degrees Celsius, to achieve thermal remodeling without charring. This range is lower than conventional RF ablation systems for cardiac applications, which typically exceed 100 degrees Celsius, thereby reducing collateral tissue damage and coagulum formation.

In some embodiments, a user interface of any of the systems described herein may display one or more of: a temperature, an impedance, and/or a power. The temperature, impedance, and/or power may be per the electrode array or one or more zones of the electrode array or independent of the electrode array or zones. The display may be in real-time, in some instances, for determining whether the settings of the generator may be changed or maintained. In some embodiments, the displaying of information may be on demand or after a procedure.

In some embodiments, the expandable support structure may be one or more expandable balloons and/or a scaffold. The scaffold may include one or more struts. The scaffold may comprise or be formed of a shape memory material to maintain a substantially even separation of the electrode array when in an expanded state. In some embodiments, the one or more expandable balloons may be formed of or made of compliant material (e.g., silicone, polyurethane, etc.), semi-compliant material (e.g., polyether block amide, nylon, polyurethane, etc.), or non-compliant material (e.g., nylon, polyester, polyethylene terephthalate, etc.). In some embodiments, expansion of the expandable support structure may be high pressure expansion. The expansion pressure may be about 4 atm to about 20 atm; about 10 atm to about 30 atm; about 10 atm to about 15 atm; at least about 5 atm; at least about 10 atm; etc. The expandable support structure may expand to a diameter of about 4 mm to about 20 mm; about 4 mm to about 18 mm; about 5 mm to about 16 mm; about 6 mm to about 15 mm; about 7 mm to about 14 mm; about 12 mm to about 16 mm; about 13 mm to about 15 mm; etc. The expandable support structure may be expanded using a fluid, for example a liquid such as saline or contrast or a gas such as carbon dioxide, nitrous oxide, or helium.

A method of treating a target vessel or a restenotic implantable device may include navigating a tissue modification device to a target site; inflating an expandable support structure to expand one or more electrodes in an electrode array at least partially surrounding the expandable support structure to create an about evenly separated electrode array and/or electrode zones of the electrode array; and activating the about evenly separated (i.e., consistently spaced) electrodes of the electrode array and/or electrode zones to deliver energy to tissue at the target site.

In some embodiments, a tissue modification device may be reusable such that the device may be deployed, contracted, and re-deployed any number of times to cover an entirety of a lesion and/or to achieve proper positioning for lesion treatment associated with an implantable device and/or a vessel.

In some embodiments, positioning of the tissue modification device relative to the implantable device and/or relative to one or more lesions may be facilitated by a handle. The handle may include one or more indicia that correspond to one or more indicia on the tissue modification device, such that an orientation of the tissue modification device may be tailored for a particular implantable device, vasculature, lesion length, lesion depth, and/or asymmetry.

In some embodiments, navigating to the target site may be based on fluoroscopic guidance. In some embodiments, the fluoroscopic guidance may also be utilized to determine orientation of the system at the target site. In some embodiments, the handle may also be used to physically rotate or re-orient the device. Additionally, or alternatively, in some embodiments, selective activation of one or more zones of RF fields may be managed to target lesions within a target site.

In some embodiments, the methods may further include retracting an outer sheath to reveal the expandable support structure and/or about evenly separated electrode array and/or electrode zones.

In some embodiments, the target site may be a restenotic implantable device, for example a stent. The target site may be an implantable device with one or more depositions of collagen and/or fibrin within a portion of a body of the implantable device. The target site may be an implantable device with one or more lesions such as clots or thrombi at least partially within a portion of the implantable device. The target site may be a deep vein thrombus site. The target site may be in an iliac vein. The target site may be in a femoral vein. The target site may be in a deep femoral vein. The target site may be in a common femoral vein. The target site may be in a posterior tibial vein, an anterior tibial vein, a peroneal vein, or a popliteal vein. The target site may be in a subclavian vein, an axillary vein, or a brachial vein. The target site may be in an inferior vena cava, an ovarian vein, or a renal vein. In some embodiments, the target site may be accessed from a femoral vein, a radial vein, or a jugular vein.

In some embodiments, the method may further include contracting (e.g., deflating) the expandable support structure and/or re-sheathing the expandable support structure and the electrode array and/or electrode zones.

In some embodiments, the expandable support structure is contracted (e.g., deflated), re-sheathed, and removed, effectively removing lesions from the implantable device lumen and restoring venous outflow.

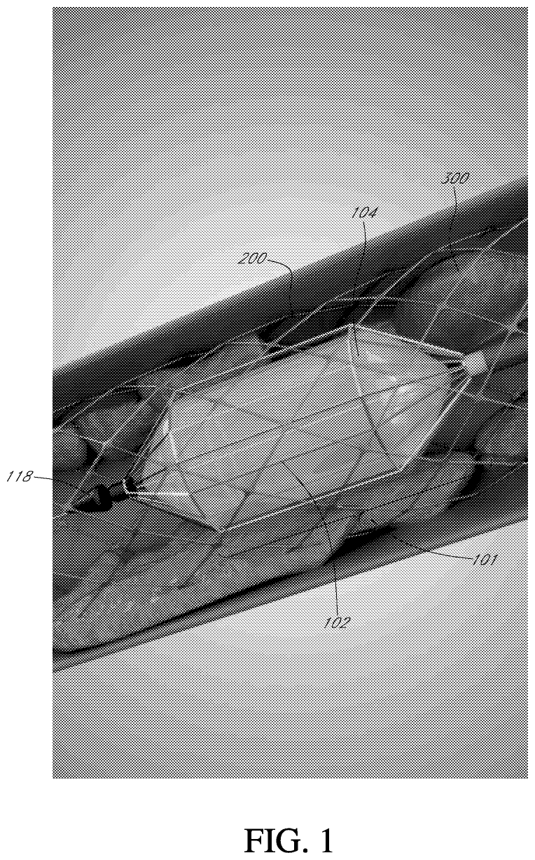

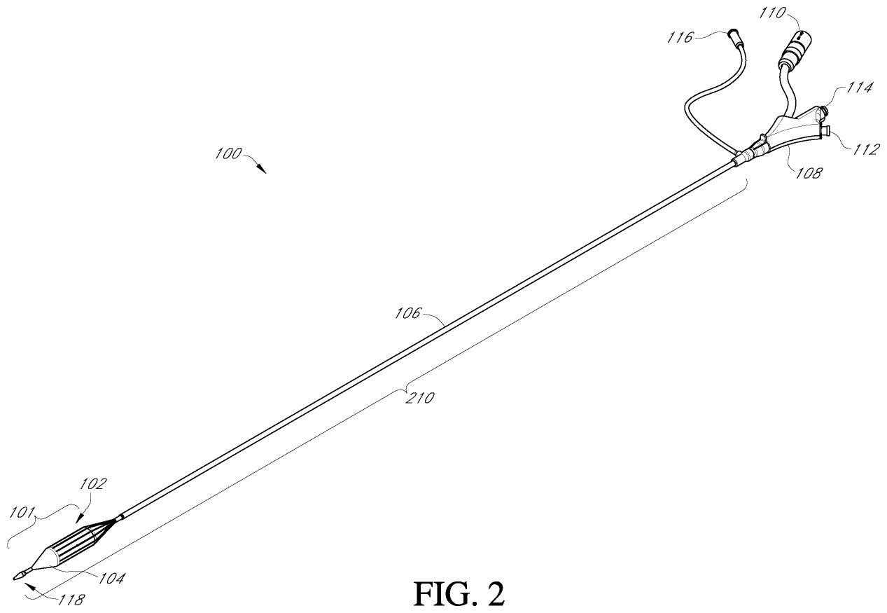

As shown in FIG. 1 illustrates a perspective view of an embodiment of a tissue modification device 101 for a target tissue 300. In some embodiments, the tissue modification device 101 is guided to the target tissue site 300 having lesions within an implantable device 200 (e.g., stent). The tissue modification device 101 may utilize an electrode array 102 supported by an expandable support structure 104 to generate an energy field for modification of the target tissue. In some embodiments, the electrode array 102 may be printed on the expandable support structure 104. In some embodiments, the electrode array 102 may be associated with a scaffold that is expandable by the expandable support structure 104. In some embodiments, the electrode array 102 may be associated with an expandable scaffold without an expandable support structure 104. In some embodiments, the elongate body 210 (see, e.g., FIG. 2) may include a shaft with sufficient flexural rigidity and torsional response to ensure delivery to the target tissue 300 and terminate with an atraumatic tip 118 to prevent injury as the elongate body 210 is guided toward the target tissue 300. The elongate body 210 may define a lumen for receiving a guiding device or other elongate member therethrough. A sidewall of the elongate body 210 may define one or more channels for routing fluid, electrical conductors, and the like to the tissue modification device 101.

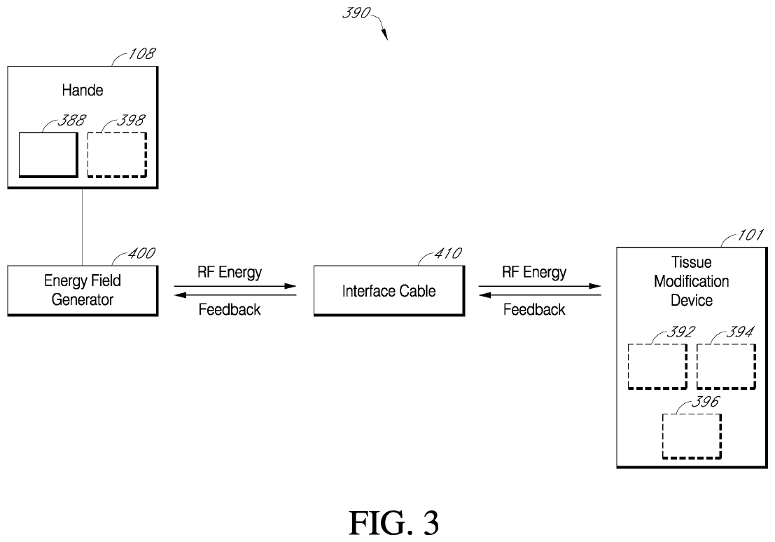

FIG. 2 illustrates a perspective view of an embodiment of a system for debulking of a target tissue. In some embodiments, the system 100 includes tissue modification device 101 and elongate body 210 working together to compress lesions and generate an energy field to debulk (e.g., densify) the target tissue in the implantable device (see, e.g., FIG. 1). The tissue modification device 101 may include an expandable support structure 104 supporting an electrode array 102 connected to a field generator (see, e.g., energy field generator 400 in FIG. 3). The energy field generator 400 is electrically coupled to the electrode array 102 through electrical connector port 110 and interface connector(s) (see, e.g., interface connector(s) 410 in FIG. 3) to provide an electrical current to the electrode array 102 and generate an energy field.

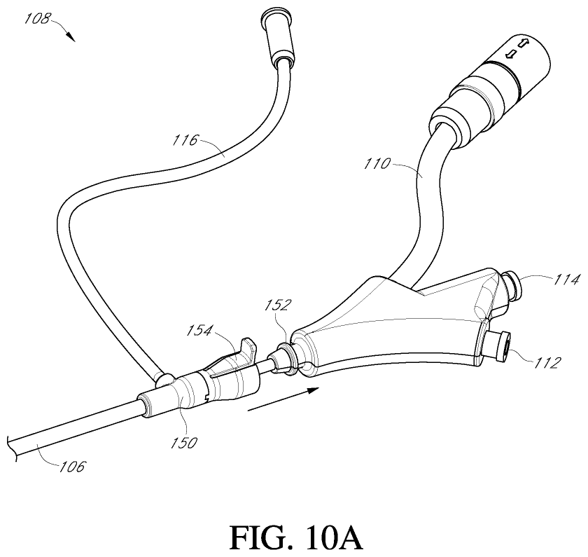

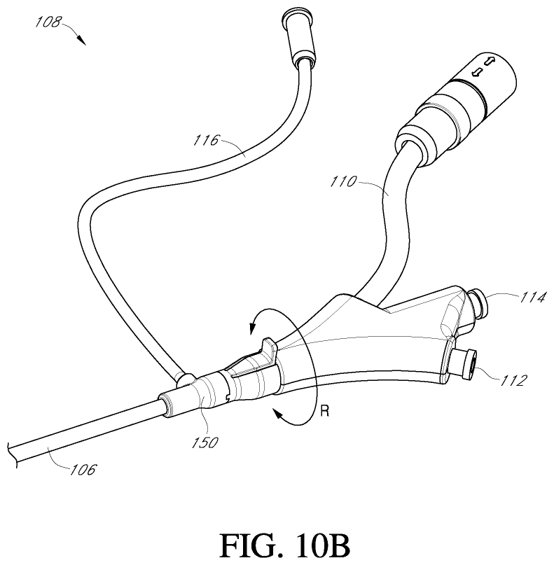

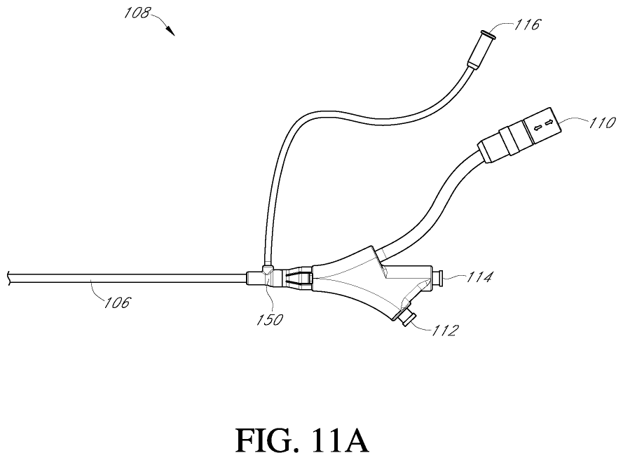

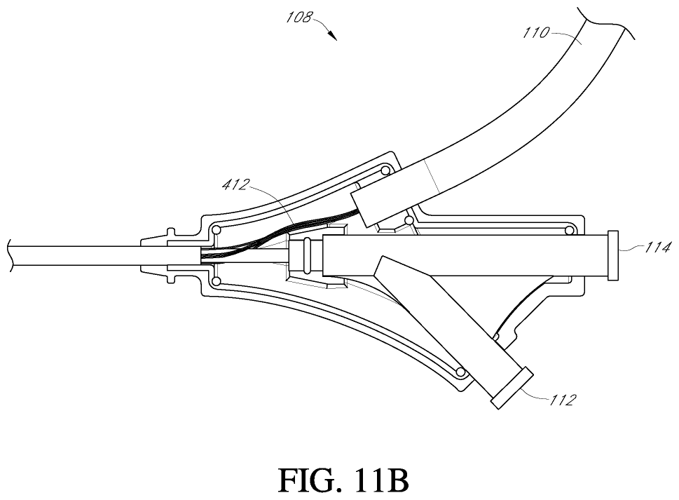

In some embodiments, the system 100 includes the elongate body 210 including a handle 108 defining one or more channels (as shown in FIGS. 10A-11B) with various controls to parts of the system 100, an outer sheath 106, and an atraumatic tip 118. In some embodiments, the one or more channels of the handle 108 may include an electrical connector port 110 for the interface connector(s) 410, an expandable support structure port 112 for controlling expansion and contraction of the expandable support structure 104, a guidance control port 114 for control of a guidewire to navigate the tissue modification device 101 to the target tissue site 300, and sheath flush port 116 for providing contrast at a target tissue or generally during a procedure (e.g., during navigation, removal, etc.).

In some embodiments, the expandable support structure 104 may include a balloon, a scaffold, and/or other structure that is capable of expansion and contraction for removal and redeployment. In some embodiments, a scaffold may include a shape memory material such as Nitinol, shape-memory polymers, shape-memory ceramics, shape-memory hydrogels, or the like. In some embodiments, the scaffold may expand and/or contract without other expandable support. In some embodiments, the balloon or other expandable support may support the electrode array 102 directly. In some embodiments, the balloon or other expandable support may support the scaffold and aid in its expansion and/or contraction and the electrode array 102 may be associated with or otherwise coupled to the scaffold. In some embodiments, the expandable support structure 104 maintains about even separation between the electrodes of the electrode array 102 to generate even energy field(s). Even separation may include a substantially equal distance or spacing between adjacent electrodes or zones of electrodes. The distance may be a linear distance or may be a distance that lies on a curve (i.e., lies on a circumference).

In some embodiments, the electrodes may include bipolar or monopolar electrodes. In some embodiments, the electrodes may include a part of the scaffold. For example, for a scaffold that is shape memory material such as Nitinol, a portion of the Nitinol material may be gold plated to act as one or more electrodes of the electrode array that may be electrically conductive and connected to an energy field generator. In some embodiments, the splines (e.g., electrodes) of the electrode array 102 have a thickness of about 0.5 mm to about 2 mm; about 0.75 mm to about 1 mm; about 1 mm to about 2 mm; about 0.5 mm to about 1.5 mm; about 0.75 mm to about 1.25 mm; etc. In some embodiments, the energy field(s) may include RF fields, microwave, pulsed field ablation, or other methods of tissue modifying energy field(s) for debulking the target tissue.

FIG. 3 illustrates a box diagram of an embodiment of a system 390 for debulking a target tissue. In some embodiments, the system 390 may include an energy field generator 400, one or more interface connectors 410, and an embodiment of a tissue modification device 101. In some embodiments, the interface connectors 410 may provide both electrical current from the energy field generator 400 (e.g., RF field generator) to the electrode array 102 of the tissue modification device 101 and return feedback information for a feedback loop from sensors such as temperature (e.g., thermocouple), impedance, and/or power output information to determine whether the energy field generation output should be adjusted to reduce or increase the temperature, reduce or increase the impedance, and/or reduce or increase the power utilization within the operating (i.e., modification) range to maintain energy delivery within a therapeutic range. For example, the therapeutic range may be between about 40 degrees Celsius and about 85 degrees Celsius or about 60 degrees Celsius to about 85 degrees Celsius. In some embodiments, an optional impedance sensor 392 may be disposed on the tissue modification device 101 at or adjacent to an electrode of the electrode array 102. The impedance sensor 392 may provide feedback regarding impedance on the electrode and determine whether the energy field output needs to be increased or decreased by maintaining impedance information (i.e., therapeutic ranges) between about 30 Ohms and about 200 Ohms; about 40 Ohms and about 180 Ohms; about 50 Ohms and about 150 Ohms; about 30 ohms to about 600 ohms; about 30 ohms to about 1,000 ohms; about 100 ohms to about 600 ohms; about 200 ohms to about 600 ohms; about 300 ohms to about 600 ohms; about 400 ohms to about 600 ohms; about 500 ohms to about 600 ohms; about 600 ohms to about 700 ohms; about 600 ohms to about 800 ohms; about 600 ohms to about 700 ohms; about 600 ohms to about 800 ohms; about 600 ohms to about 900 ohms; etc. In some embodiments, optional current sensor(s) 396 may be used to provide feedback regarding power utilization may be used to determine whether to maintain energy field output at a therapeutic range to maintain the energy field outputs on the target tissue(s).

In some embodiments, the feedback information may be provided at an optional graphical user interface 398. The graphical user interface 398 may be associated with a handle 108 of the system 390 or associated with a computing device communicatively coupled with the system 390. The graphical user interface 398 may be communicatively coupled (e.g., wired or wireless connection) to a controller 388 (e.g., microprocessor, programmable logic controller, application-specific gate array, etc.) for managing the energy field generators, one or more sensors of the system 390, and/or a separate computing device. The controller 388 may be associated with the handle 108, a computing device, the energy field generator 400, or another communicatively coupled device. In some embodiments, the graphical user interface 398 displays or presents the feedback information so that the generated energy field may be altered to account for the feedback.

In some embodiments, a different energy field generator 400 may be utilized to generate electrical current for each channel or zone of the electrode array 102. In some embodiments, a single energy field generator 400 may generate electrical current for some or all the channels or zones of the electrode array 102. In some embodiments, a controller 388 or one of the energy field generators may be utilized to manage energy field generation from the one or more energy field generators 400. For example, a treatment of the target tissue may include selectively activating one or more zones to ablate (i.e., treat) lesions adjacent to the activated one or more zones of the electrode array 102. The treatment may also include deactivating the one or more of the one or more zones, contracting and/or reorienting the tissue modification device 101, and reactivating one or more of the one or more zones to treat the lesion(s) with another energy field. In some embodiments, the controller 388 or the energy field generator 400 may also adjust the energy field power output to generate another energy field to treat the lesion without reorienting the tissue modification device 101.

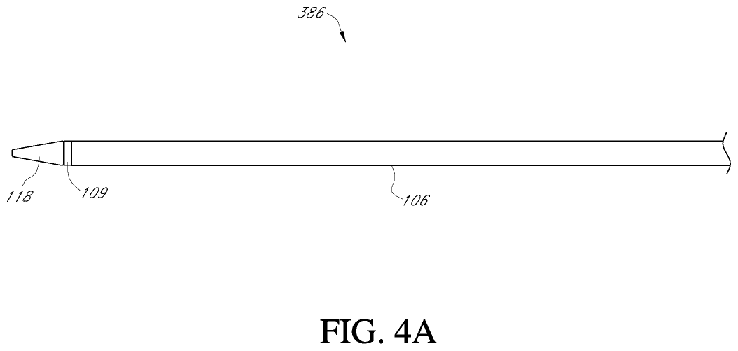

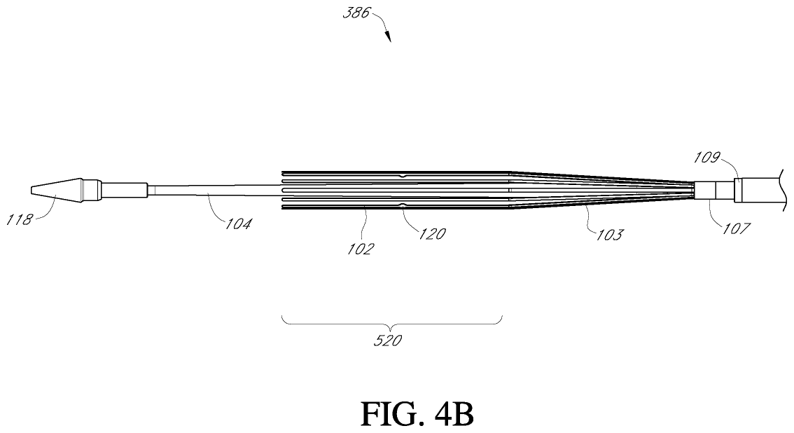

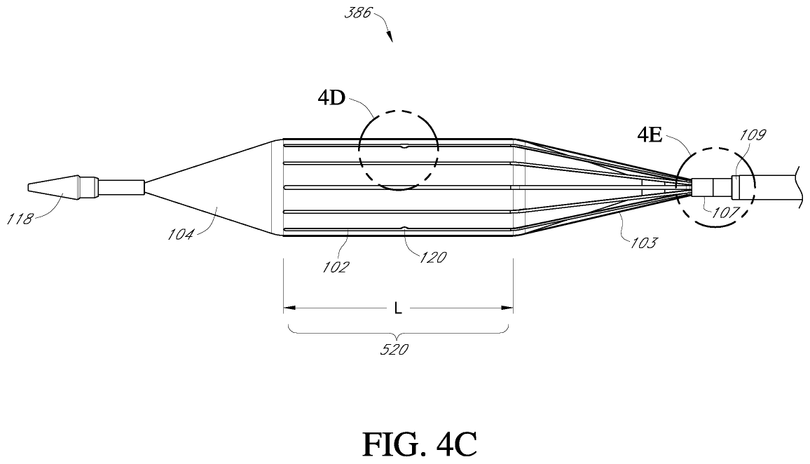

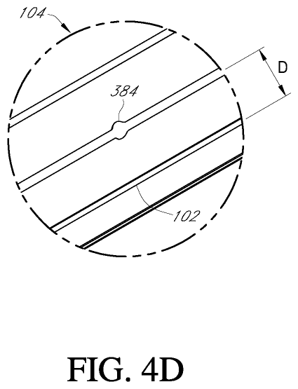

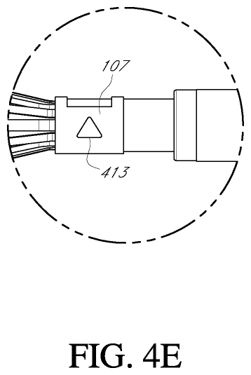

FIGS. 4A-8A show various embodiments of tissue modification devices with like components being labeled with similar or the same element numbers.

FIG. 4A illustrates a side view of an embodiment of a distal end 386 of a system with an outer sheath 106 axially advanced to cover and optionally protect the tissue modification device. The elongate body 210 (shown in FIG. 2), that is co-axially disposed within the outer sheath 106, terminates at the distal end 386 with an atraumatic tip 118 to aid in guiding the tissue modification device through the vasculature of the patient. In some embodiments, the elongate body 210 may be attached to a cuff 109 (see, e.g., FIG. 4B) that may comprise or be formed of a more rigid material, than the elongate body 210, to enable recapture of an expanded expandable support structure 104 either after energy field treatment or in order to re-orient or re-position the tissue modification device. In some embodiments, the outer sheath 106 and/or elongate body 210 may retain or provide sufficient flexural rigidity and torsional response to ensure safe delivery of the tissue modification device to the affected patient area while enough flexibility to travel through the vasculature (e.g., ilia) and/or other parts of the body to reach the target tissue. For example, the cuff 109 may comprise or be formed of a polyetheretherketone (PEEK) material with a rounded tip that is more rigid than the outer sheath 106 and/or elongate body 210 to allow the electrodes to be recaptured without catching on the outer sheath 106. Although PPEK is described herein, other materials may include, but not be limited to, Polyetherketoneketone, Polyetherimide, Polyphenylsulfone, Polysulfone, Polyphenylene sulfide, Liquid Crystal Polymer, and the like. The outer sheath 106 and/or elongate body 210 may comprise of a polyfluoroalkyl (PFAS) material to allow some flexure when directing the tissue modification device 101 to the target tissue, but with enough rigidity and torsional response to allow orientational manipulation by the handle 108. Although PFAS is described herein, other materials may include, but not be limited to, polyether block amide, nylon, Polyimide, Polyvinylpyrrolidone, Polyacrylamide, Polyethylene glycol, Poly(2-oxazolines), and the like. In some embodiments, the outer sheath 106 may axially translate proximally relative to the tissue modification device to unsheathe the tissue modification device and allow the expandable support structure 104 space to expand.

In some embodiments, as shown in FIG. 4E, navigating to the target site may be based on fluoroscopic guidance and utilized to determine orientation of the tissue modification device at the target tissue site. In some embodiments, an asymmetric orientation indicator 413 to show where a first zone may be oriented may be indicated on the outer sheath 106, expandable support structure, a cuff 107, and/or scaffold. For example, an asymmetric orientation indicator, such as by radiopaque laser cut platinum band(s) showing an arrow and/or stripe and/or other directional indicator may be utilized to indicate the orientation of the tissue modification device prior to expansion.

FIG. 4B illustrates a side view of the embodiment of the distal end 386 of the system of FIG. 4A with the outer sheath 106 pulled back (i.e., retracted) relative to the cuff 109 to expose the expandable support structure 104 and electrode array 102. In some embodiments, the removed outer sheath 106 exposes an electrical conductor 107 connecting the electrode array 102 to the interface connector(s) 410 that are disposed in the elongate body of the system. In some embodiments, the electrical conductor 107 includes electrical connections between interface connector(s) 410 connecting to the energy field generator 400 and each of the one or more electrodes of the electrode array 102 and each of the sensor(s) 120. In some embodiments, the electrode array 102 is connected to the electrical conductor 107 with insulated ramps 103 or struts. The insulated ramps 103 may include the one or more electrodes covered with insulation to prevent energy field generation outside of the apex section 520 (i.e., the section of the expandable support structure 104 that is at the maximum expanded volume, for example, a maximum expanded circumference of a balloon), when expanded, of the expandable support structure 104. Although insulated ramps 103 are shown at a proximal end of the electrode array 102, one of skill in the art will appreciate that insulated ramps may also be disposed on a distal end of the electrode array 102 to connect the distal end of the electrode array to a distal end of the tissue modification device (e.g., at or adjacent to tip 118).

In some embodiments, the electrical conductor 107 may electrically couple one positive wire for two positive splines (i.e., electrodes); one or more wires for each negative spline; and at least one wire for each sensor (e.g., thermocouple, impedance, etc.).

FIG. 4C illustrates a side view of the embodiment of the distal end 386 of the system of FIG. 4A with the expandable support structure 104 expanded to support the electrode array 102. As shown, in some embodiments, the apex section 520 retains the one or more electrodes of the electrode array 102. In some embodiments, when expandable support structure 104 is expanded, apex section 520 of the electrode array 102 may comprise splines of about even spacing along an axial length L. The axial length L may comprise of any suitable length that may extend between at least one axial section of the stent 200. In other words, the axial length L may treat a stent 200 of the same length in a single treatment. However, the tissue modification device 101 may often need to be redeployed a plurality of times along each axial section of the stent 200 to treat an entire length of a stent 200 for restenosis. For example, for a tissue modification device 101 with an axial length of about 40 mm and stent 200 with a length of about 200 mm, the tissue modification device 101 may be deployed (and redeployed) at least five times to cover the length of the stent 200. As shown in FIG. 4D, when expanded, each electrode 384 of the electrode array is spaced at a substantially equal distance D from an adjacent electrode or, said another way, adjacent electrodes are spaced evenly apart by a distance D. Distance D may be based on an expansion diameter or circumference of the expandable support structure 104.

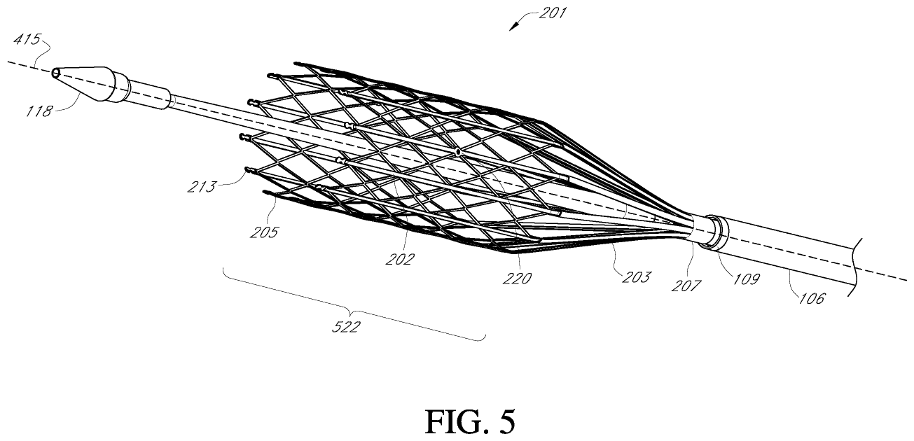

FIG. 5 illustrates a perspective side view of an embodiment including an expandable support structure 104 that comprises a scaffold 205 without a balloon. The scaffold 105 maintains the electrode array 202 at an even spacing, similar to what is shown in FIG. 4D, when the expandable support structure 104 is in an expanded state. In some embodiments, the scaffold 205 may include a plurality of struts. In some embodiments, the scaffold 205 may include an open cell structure. In some embodiments, the scaffold 205 may include a closed cell structure. In some embodiments, the scaffold 205 may include struts in a mesh or lattice-like structure where the struts of the scaffold 205 may run substantially parallel to longitudinal axis 415 over at least a part of the scaffold 205 and may include a substantially even spacing between adjacent struts. Alternatively, the struts of the scaffold may run substantially perpendicular to longitudinal axis 415. In some embodiments, the scaffold 205 may include one or more radiopaque markers 213 on a distal end of the scaffold 205. In some embodiments, the scaffold 205 may terminate in an open distal end, as shown in FIG. 5, such that the scaffold 205 has a basket-like shape. In some embodiments, the electrodes of the electrode array 202 are evenly spaced when the scaffold 205 is expanded and partially run in parallel, or non-perpendicularly, axially along the tissue modification device 201, relative to the longitudinal axis 415, to maintain the even spacing in the apex section 522. In other words, in some embodiments, the electrodes may be parallel to a longitudinal axis 415 of the device, with or without a scaffold or printed on an expandable support structure. In some embodiments, the substantially equal spacing of the electrodes may be substantially parallel to a longitudinal axis of the device. In some embodiments, the substantially equal spacing of the electrodes may be substantially non-perpendicular to a longitudinal axis of the device. In some embodiments, the substantially equal spacing of the electrodes may be substantially perpendicular to a longitudinal axis of the device, for example, such that the electrodes are along a diameter or circumference of the device. As shown, the insulated ramps 203 meet at electrical conductor 207. In some embodiments, one or more of the electrodes of the electrode array 202 may include sensor(s) 220.

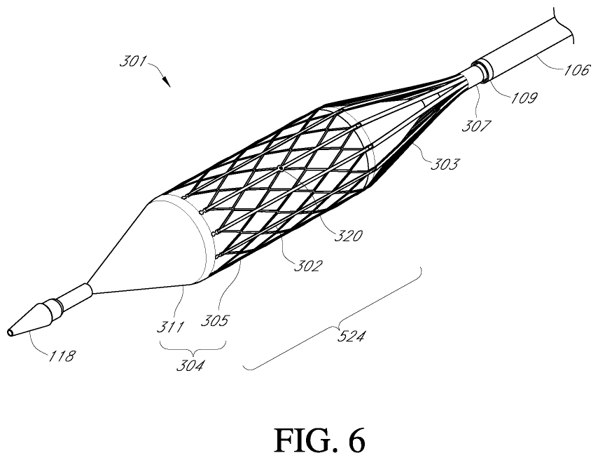

FIG. 6 illustrates a perspective side view of an embodiment with the electrode array 302 partially embedded into, integrated into, or mounted on a portion of a scaffold 305 of an expandable support structure 304. The expandable support structure 304 may also include a balloon 311 that expands to support the expanded state of the expandable support structure 304 and retain the even spacing of the one or more electrodes of the electrode array 302 when the expandable support structure 304 is expanded. In other words, in the expanded state, the electrodes of the electrode array 302 may run in parallel axially along the length of the apex section 524 of tissue modification device 301. In some embodiments, the scaffold 305 may include a plurality of struts. In some embodiments, the scaffold 305 may include an open cell structure. In some embodiments, the scaffold 305 may include a closed cell structure. In some embodiments, the scaffold 305 may include a mesh or lattice-like structure. In some embodiments, the scaffold 305 may terminate in an open distal end, as shown in FIG. 6, such that the scaffold 305 has a basket-like shape. In some embodiments, the insulated ramps 303 are electrically coupled to the electrical array 302 and the electrical connector 307. In some embodiments, one or more of the electrodes of the electrode array 302 may include sensor(s) 320.

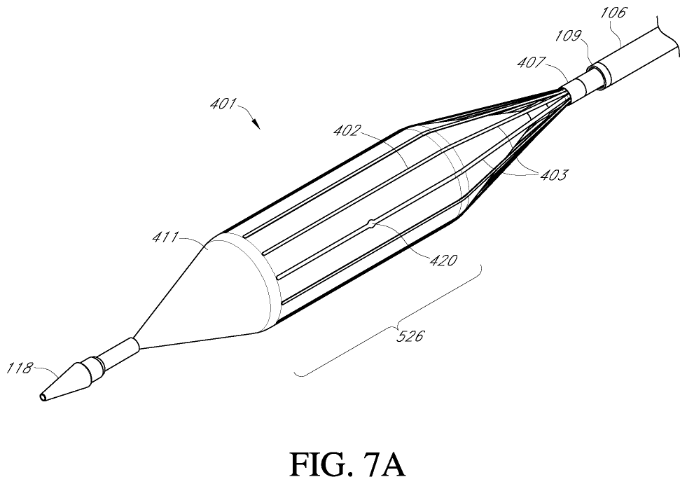

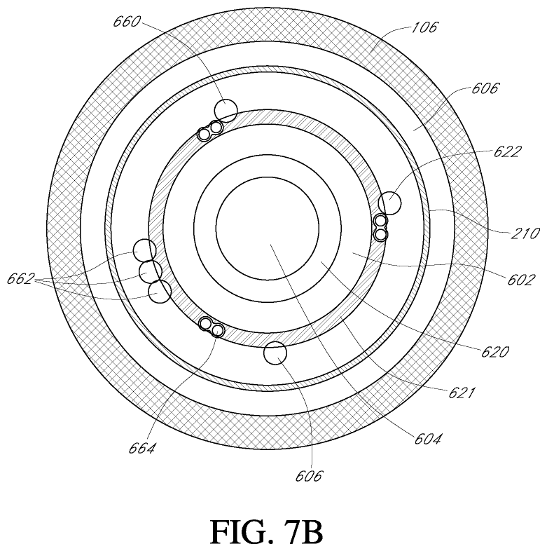

FIG. 7A illustrates a perspective view of an embodiment of a distal end of a system with the electrode array 402 directly printed on the balloon 411 of the expandable support structure. For example, the electrode array 402 may be printed on a surface of the balloon 411 using laser printing, aerosol jet printing, conductive polymer coatings, stretchable metallic inks, liquid metal printing, thermal transfer printing, screen printing of stretchable conductors, and the like. When in an expanded state, the electrodes of electrode array 402 may be evenly spaced (e.g., run in parallel or at least non-perpendicularly) in the apex section 526 along the axial length (see also, e.g., axial length L of FIG. 4C) of the tissue modification device 401. In some embodiments, the insulated ramps 403 are electrically coupled to the electrical array 402 and the electrical connector 407. In some embodiments, one or more of the electrodes of the electrode array 402 may include sensor(s) 420.

As shown in FIG. 7B illustrates a cross-sectional view of a distal end of a tissue modification device showing the one or more electrodes of the electrode array 102. FIG. 7B also references channels of the handle 108 (see, e.g., in FIGS. 2, 10A-10B, and 11A-11B) that are used to manipulate the system 100. For example, the expandable support structure port 112 (FIG. 10A) is communicatively coupled to an expandable support structure lumen 602 for filling a balloon or expanding a scaffold, a guidance control port 114 (FIG. 10A) communicatively coupled to a guidance lumen 604 for controlling a guiding mechanism (e.g., guidewire) to direct the tissue modification device to the target tissue site, and a sheath flush port 116 (FIG. 10A) communicatively coupled to an outer sheath lumen 606 for flushing the lumen of the outer sheath 106 to inject contrast media between the over sheath 106 and the elongate body. In some embodiments, the additional layers may exist to insulate manipulating portions of the system 100. For example, an inner shaft 620 may define the guidance lumen 604 that receives the guidance mechanism. The space between inner shaft 620 and outer shaft 621 defines the fill lumen 602 for the expandable support structure. Additionally, the elongate body 210 may include an insulating wrap 622 to cover the wires 660, 662, 664 and connected electrical conductor 107 to prevent flushes through the lumen of the outer sheath lumen 606 from affecting the electrical system. In some embodiments, the insulating wrap 622 may comprise or be formed of a heat shrink polymer or plastic, for example PEBAX or similar. In some embodiments, one or more wires 660, 662, 664 may be electrically coupled to splines of the electrode array 102. Wire 660 may be coupled to, in bipolar electrodes, one or more positive splines. Wires 662 may be coupled to one or more negative splines and may utilize three wires to reduce heat from carrying an electrical current while retaining a slim profile and reducing thickness of the system. Wire 664 may be coupled to a sensor such as a temperature sensor or impedance sensor to receive real-time feedback (i.e., monitor) for managing the energy field generation. In some embodiments, the wires 660, 662, 664 may be similar in thickness, but it is contemplated that different wires may be utilized for each spline as long as current carrying capacity and feedback may be retained without heat from the wires affecting the generated energy field and target tissues.

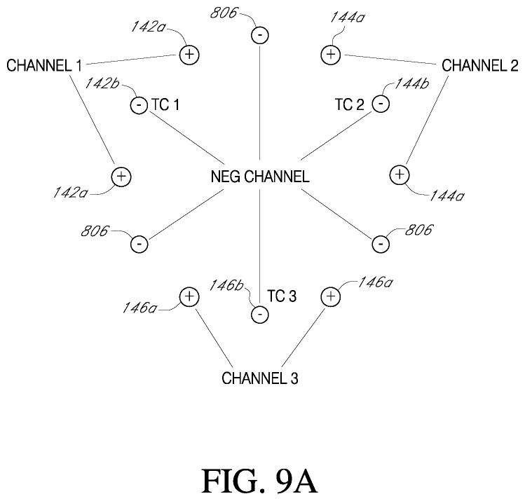

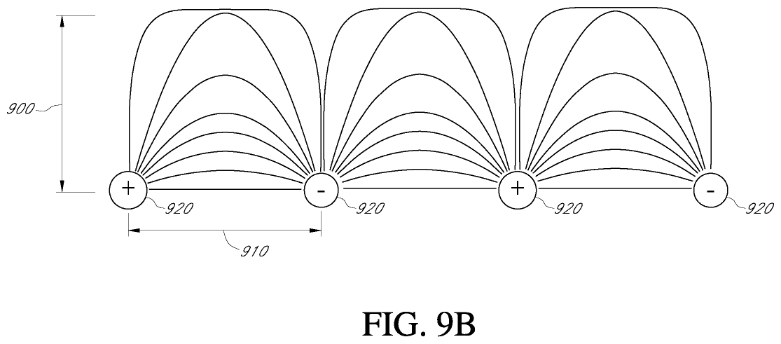

FIG. 8A illustrates a perspective view of an embodiment of a distal end of a system 800 with an expandable support structure 804 expanded to support an electrode array 802 that is generating a plurality of energy fields or zones 840a, 840b, 840c. In some embodiments, the sensor(s) 820 are centered between the electrodes of the three energy field channels 840a, 840b, 840c or may be co-located with one or more electrodes. Each of the energy fields or zones 840a, 840b, 840c, including two positive electrodes and three negative electrodes, act to create a bipolar energy field. In other words, a set of three electrodes particular to each channel and two shared negative electrodes are utilized for each energy field zone 840a, 840b, 840c. As shown, and more clearly shown in FIG. 9A, each shared end electrode 806 is a negative electrode. Between the shared end electrodes 806 are three electrodes that include a negative electrode with a sensor between two positive electrodes.

FIG. 8B illustrates a transverse cross-sectional view of the tissue modification device of the system 800 of FIG. 8A. In this illustration, 12 bipolar electrodes and three zones 840a, 840b, 840c are shown. Each electrode of the electrode array 802 is shown as generating an energy field with the neighboring electrodes. The expandable support structure 804 as expanded, and shown in FIG. 8B, generates an angular distribution of the energy zones 840a, 840 b, 840 c. As shown in FIG. 8B, each zone is about 120 degrees of the circumference of the expandable support structure 804. Although three zones are shown, any number of zones are contemplated herein. For example, one zone may be present, one to five zones, 3 to 5 five zones, five to ten zones, six to twelve zones, etc.

FIG. 9A illustrates a channel map of a cross-section of the tissue modification device showing the related energy field channels utilized to generate an energy field. Each channel represents an electrode of the electrode array 102. For example, the energy field channels or zones 840a, 840b, 840c of FIGS. 8A-8B may include a pair of positive channels 142a, 144a, 146a, a negative channel 142b, 144b, 146b with a sensor, and shared negative end electrodes 806. Each negative channel 142b, 144b, 146b may include a sensor such as a temperature sensor (as shown by sensor 820 of FIG. 8A). Thus, at least in some embodiments, nine channels exist for three energy field channels 140a, 140b, 140c. In some embodiments, more channels may exist to create more than three energy field channels or zones 840a, 840b, 840c, or fewer channels may exist to create less than three energy field channels or zones.

FIG. 9B illustrates a linear channel map describing the depth 900 of the energy field generated by the electrodes and spacing 910 between the electrodes 920. In some embodiments, the spacing 910 may be utilized to determine the depth 900 of the energy field. For example, an about 4 mm spacing between bipolar electrodes 920 may generate an energy field with a depth of about 4 mm.

FIGS. 10A-10B illustrate perspective views of an embodiment of a handle 108 for a tissue debulking system. In some embodiments, the outer sheath 106 may be coupled to a handle interface 150 to lock (e.g., clip) the outer sheath 106 to the handle 108 by clipping the handle interface 150 at clip 154 to the locking ring 152. As shown in FIG. 10B, the locking ring 152 allows docking of the outer sheath to the handle 108 using handle interface 150 once the outer sheath is retracted. Handle interface 150 may be rotated using rotation R to couple the outer sheath 106 to the handle 108. As described elsewhere herein, the handle 108 further includes an expandable support structure port 112 for controlling expansion and contraction of the expandable support structure, a guidance control port 114 for control of a guidewire to navigate the tissue modification device, sheath flushing port 116, and an electrical connector port 110 for the interface connector(s).

FIGS. 11A-11B illustrate a side and cross-sectional view, respectively, of a handle 108 with one or more channels for electrical connections, guide mechanisms, over sheath flush port, and expandable support structure controls. In some embodiments, the channels of the handle 108 include an electrical connector port 110 for the interface connector(s) 410 (of FIG. 3) to electrically connect to an energy field generator 400 (of FIG. 3), an expandable support structure port 112 for controlling expansion and contraction (e.g., using a fluid) of the expandable support structure, a guidance control port 114 for control of a guidewire to navigate the tissue modification device to the target tissue site, and sheath flush port 116 for managing the removal and covering of the tissue modification device and flushing the over sheath 106 to inject contrast media between the over sheath 106 and the electrical conductor. Wires 412 may be provided to electrically connect the energy field generator 400 to the electrode array of the tissue modification device.

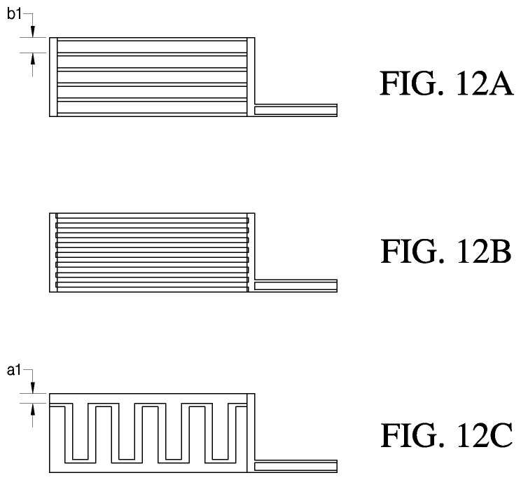

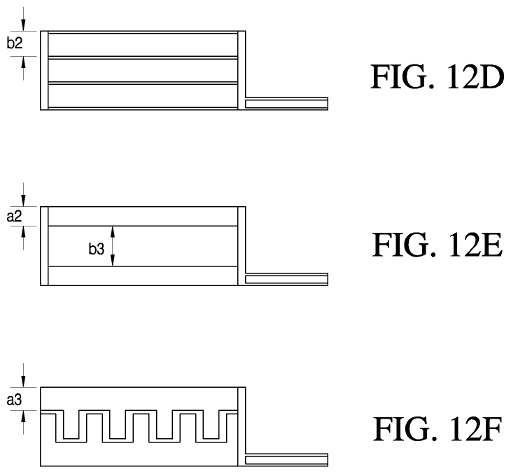

FIGS. 12A-12F illustrate top views of various embodiments of electrode arrays 102 that may be utilized to generate tissue modification for a target tissue 300. FIG. 12A shows an array of straight electrodes with spacing b1. Spacing b1 may be about 2 mm to about 6 mm; about 2 mm to about 3 mm; about 2 mm to about 4 mm; about 2 mm to about 5 mm; about 3 mm to about 5 mm; about 4 mm to about 6 mm; etc. FIG. 12B shows an array of electrodes arranged in a serpentine shape. FIG. 12C shows an array of electrodes with a square waveform shape corresponding to the electrode next to it and an electrode thickness a1. Spacing a1 may be about 0.4 mm to about 1.25 mm; about 0.5 mm to about 1.0 mm; about 0.75 mm to about 1.25 mm; about 1 mm to about 1.25 mm; about 0.75 mm to about 1 mm; about 0.5 mm to 0.75 mm; about 0.6 mm to about 0.8 mm; etc. FIG. 12D shows an array of straight electrodes with a wider, than FIG. 12A, but even, spacing b2. Spacing b2 may be about 2 mm to about 6 mm; about 2 mm to about 3 mm; about 2 mm to about 4 mm; about 2 mm to about 5 mm; about 3 mm to about 5 mm; about 4 mm to about 6 mm; etc. FIG. 12E shows an array of straight electrodes with a wide, but even, spacing b3, and an electrode thickness a2. Spacing b3 may be about 2 mm to about 6 mm; about 3 mm to about 5 mm; about 4 mm to about 5 mm; etc. Thickness a2 may be about 0.4 mm to about 2 mm; about 0.5 mm to about 1 mm; about 1 mm to about 2.0 mm; about 1.5 mm to about 2.0 mm; about 0.4 mm to about 0.8 mm; etc. FIG. 12F shows an array of square waveform shaped electrodes with thickness a3, thicker than the electrodes of FIG. 12C. Thickness a3 may be about 0.4 mm to about 2 mm; about 0.5 mm to about 1 mm; about 1 mm to about 2.0 mm; about 1.5 mm to about 2.0 mm; about 0.4 mm to about 0.8 mm; etc. The shape and thickness of these electrodes may result in different energy fields that may reach treatment depths of at least 4 mm and are considered potential designs for the electrode array 102 as long as the energy fields generated may treat target tissues between about 1 mm and about 10 mm deep and be controllable (i.e., may be oriented) to therapeutically target tissue sites.

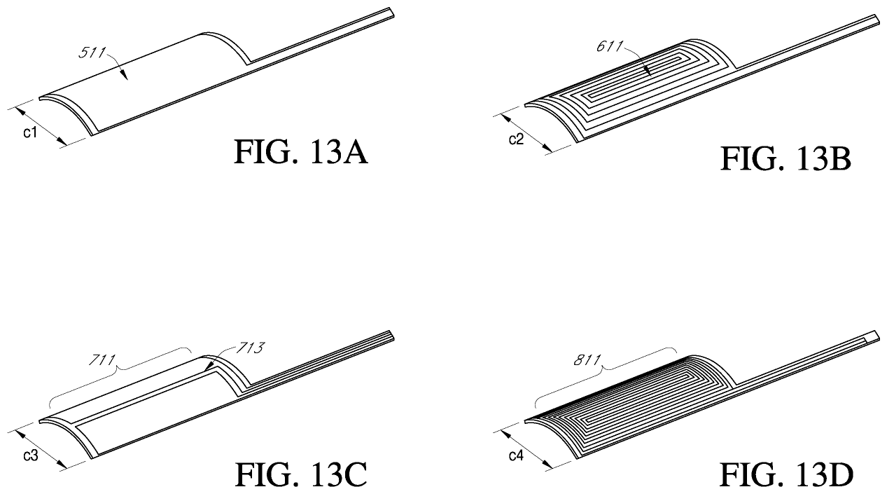

FIGS. 13A-13D illustrate perspective views of various embodiments of electrode arrays that may be utilized to generate energy field ablation for a target tissue. The electrode designs of FIGS. 13A-13D are designed to sit along a curved substrate (e.g., an expandable support structure, scaffold, or balloon), with differences in polarity (monopolar vs. bipolar) and patterning (solid vs. coil/mesh). These additional designs for the electrode arrays may include monopolar or bipolar electrode designs. Similar to the electrode array designs of FIGS. 12A-12F, these designs may be considered optional based on their capability to therapeutically treat target tissues.

FIG. 13A shows a single continuous conductive surface on a curved backing. As shown in FIG. 13A, no return electrode is integrated, a separate remote dispersive electrode may be used to complete the circuit. As shown in FIG. 13A, the conductive portion may be a solid sheet, providing uniform surface contact with a target tissue. FIG. 13A shows a monopolar electrode 511 with a width c1 wider than the electrode interface 510 and having a radius of curvature complementary to a surface of a rounded object such as an expandable support structure. Width c1 may be about 9 mm to about 30 mm; about 10 mm to about 20 mm; about 20 mm to about 30 mm; about 9 mm to about 19 mm; about 10 mm to about 15 mm; about 15 mm to about 20 mm; about 20 mm to about 25 mm; about 25 mm to about 30 mm; etc.

FIG. 13B shows a coil/mesh or serpentine trace pattern. The trace pattern of FIG. 13B may provide increased flexibility, compliance on curved or expanding surfaces, and/or a surface area without inhibiting expansion. The monopolar coiled electrode 611 of FIG. 13B may have a radius of curvature complementary to a surface of a rounded object such as an expandable support structure with width c2 and electrode interface 610. Width c2 may be about 9 mm to about 30 mm; about 10 mm to about 20 mm; about 20 mm to about 30 mm; about 9 mm to about 19 mm; about 10 mm to about 15 mm; about 15 mm to about 20 mm; about 20 mm to about 25 mm; about 25 mm to about 30 mm; etc.

FIG. 13C shows two separate conductive regions on the same curved surface. The bipolar electrode 711 of FIG. 13C allows the circuit to complete locally to confine energy delivery to a small region, reduce stray current paths, and/or provide better precision for ablation, stimulation, or sensing. The two poles are typically separated by an insulating gap 713. The bipolar electrode 711 of FIG. 13C has a width c3 wider than the electrode interface 710 and may have a radius of curvature complementary to a surface of a rounded object such as an expandable support structure. Width c3 may be about 9 mm to about 30 mm; about 10 mm to about 20 mm; about 20 mm to about 30 mm; about 9 mm to about 19 mm; about 10 mm to about 15 mm; about 15 mm to about 20 mm; about 20 mm to about 25 mm; about 25 mm to about 30 mm; etc.

FIG. 13D shows a combination of FIGS. 13B-13C. As shown in FIG. 13D, two electrodes (bipolar) are each formed using coil/mesh patterned conductive traces. The bipolar electrode 811 of FIG. 13D may provide high flexibility, conformability, localized current delivery, and/or the ability to cover more surface area without overly stiffening the structure. The bipolar coiled electrode may have a radius of curvature complementary to a surface of a rounded object such as an expandable support structure and have a width c4 and electrode interface 810. Each of the electrode array designs of FIGS. 13A-13D may define a single energy field zone, thus, to encircle a rounded object, at least a few of these electrode array designs may be used to fully encircle a rounded object (e.g., cylindrical object such as an expandable support structure). Width c4 may be about 9 mm to about 30 mm; about 10 mm to about 20 mm; about 20 mm to about 30 mm; about 9 mm to about 19 mm; about 10 mm to about 15 mm; about 15 mm to about 20 mm; about 20 mm to about 25 mm; about 25 mm to about 30 mm; etc.

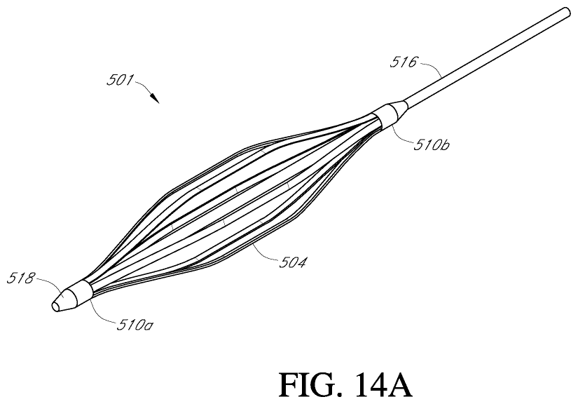

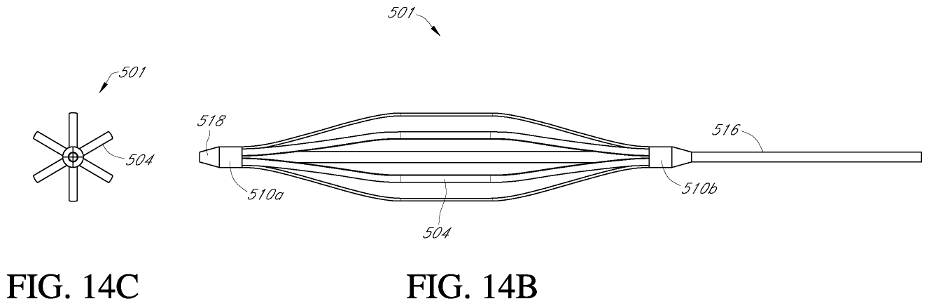

FIGS. 14A-14C illustrate a perspective view, side view, and end view, respectively, of an embodiment of a tissue modification device 501 with a distal end that may be expanded and/or contracted by shifting hubs 510a, 510b closer together to expand the scaffold 504 without other expanding mechanisms such as balloons. For example, distal hub 510a may be fixed to a distal end of shaft 516 while proximal hub 510b may axially translate along shaft 516 to expand or contract the scaffold 504. In some embodiments, the tissue modification device 501 includes an atraumatic tip 518. In some embodiments, the scaffold 504 may include a plurality of struts. In some embodiments, the scaffold 304 may include a mesh or lattice-like structure. In some embodiments, the scaffold 504 may include one or more radiopaque markers 213 (as shown in FIG. 5) disposed along its length.

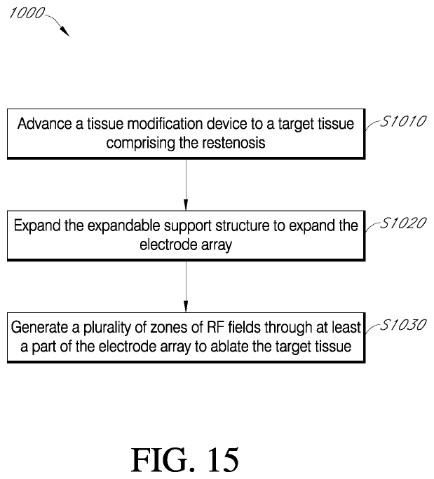

As shown in FIG. 15, an embodiment of a method 1000 for treating restenosis of an implantable device includes advancing a tissue modification device to a target tissue comprising the restenosis in block S1010, expanding the expandable support structure to expand the electrode array in block S1020, and generating a plurality of zones of RF fields through at least a part of the electrode array to ablate the target tissue in block S1030. The method functions to treat restenosis by generating an evenly spaced electrode array to target lesions within the vessel. In some embodiments, the method functions to densify the lesions and prevent and/or reduce the likelihood of restenosis. The method is used for treating restenosis but can additionally, or alternatively, be used for any suitable applications, clinical or otherwise. The method can be adapted to function for any suitable densification of lesions in vessels.

As shown in FIG. 15, an embodiment of a method 1000 for treating restenosis includes block S1010, which recites advancing a tissue modification device to a target tissue comprising the restenosis. Block S1010 functions to provide the tissue modification device to the target tissue site. The target tissue site 300 may be within a lumen of an implantable device (e.g., stent 200) in a vessel of a patient. In some embodiments, one or more radiopaque markers may be utilized to determine the orientation and/or location of the tissue modification device at the tissue site under fluoroscopy. In some embodiments, the tissue modification device may be advanced over a guidewire to direct the tissue modification device to the target tissue site. Once at the target tissue site, a handle of the system may be manipulated to rotate or orient the tissue modification device at the target tissue site and/or a determination may be made regarding how the tissue modification device is oriented and one or more zones of electrodes of the electrode array may be selected to treat particular lesions at the target tissue site. For example, lesions in particular zones may be treated by energy fields from the same zone. In some embodiments, one or more indicia on a handle of the system may be aligned with one or more indica on the tissue modification device to determine an orientation of the tissue modification device relative to the implantable device, the anatomy, and/or one or more lesions.

As shown in FIG. 15, an embodiment of a method 1000 for treating restenosis includes block S1020, which recites expanding the expandable support structure to expand the electrode array. Block S1020 functions to prepare the electrode array for a state to generate an energy field for ablating the lesions. In some embodiments, the expandable support structure may include a balloon and/or scaffold. In some embodiments, the expandable support structure may include only a balloon. For example, the electrode array may be directly printed to a surface of the balloon. In some embodiments, the expandable support structure may include only a scaffold. For example, the electrode array may be partially embedded into, integrated into, or mounted on one or more struts or components of the scaffold. In some embodiments, the expandable support structure may include both the balloon and scaffold. The electrode array may be partially embedded into, integrated into, or mounted on the scaffold, but the balloon and/or scaffold may aid in the expansion of the tissue modification device to create an outward radial or expansion force (i.e., pressure). The expansion pressure may be about 4 atm to about 20 atm; about 10 atm to about 30 atm; about 10 atm to about 15 atm; at least about 4 atm; at least about 10 atm; etc. The expansion pressure may be utilized to compress the lesion prior to densification to reduce the restenosis in the target tissue.

As shown in FIG. 15, an embodiment of a method 1000 for treating restenosis includes block S1030, which recites generating a plurality of zones of energy fields through at least a part of the electrode array to ablate the target tissue. Block S1030 functions to generate the energy field (e.g., RF field) from the expanded state of the electrode array to target the lesions. In some embodiments, when a balloon is utilized, the energy fields are insulated from affecting blood in the vessel due to the balloon (i.e., the balloon occludes the lumen of the implantable device and/or vessel lumen). The RF fields are generated to treat the target tissue at a predefined depth.

The systems and methods described herein, and variations thereof, can be embodied and/or implemented at least in part as a machine configured to receive a computer-readable medium storing computer-readable instructions. The instructions are executable by computer-executable components integrated with the system and one or more portions of the processor of the control system and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component may be a general or application-specific processor, but any suitable dedicated hardware or hardware/firmware combination can alternatively, or additionally, execute the instructions.

### EXAMPLES

Example 1. A tissue modification device for modification of a target tissue within a vessel, the tissue modification device comprising: an elongate body comprising the tissue modification device on a distal end of the elongate body, the tissue modification device comprising: an electrode array, and an expandable support structure configured to support the electrode array, wherein, when expanded, the expandable support structure is configured to space adjacent electrodes of the electrode array at a substantially equal distance, and wherein the electrode array is configured to generate one or more tissue modification energy fields around the expandable support structure when an electrical current is supplied to the electrode array.

Example 2. The tissue modification device of any one of the preceding examples, but particularly Example 1, wherein the one or more tissue modification energy fields comprise radiofrequency (RF) fields.

Example 3. The tissue modification device of any one of the preceding examples, but particularly Example 1, wherein the target tissue is within an implantable device within the vessel.

Example 4. The tissue modification device of any one of the preceding examples, but particularly Example 1, wherein the electrode array is configured to expand responsive to expansion of the expandable support structure.

Example 5. The tissue modification device of any one of the preceding examples, but particularly Example 4, wherein the electrode array is carried by a scaffold on the expandable support structure.

Example 6. The tissue modification device of any one of the preceding examples, but particularly Example 5, wherein the scaffold includes one or more struts.

Example 7. The tissue modification device of any one of the preceding examples, but particularly Example 6, wherein at least one electrode of the electrode array is carried by one or more of the one or more struts.

Example 8. The tissue modification device of any one of the preceding examples, but particularly Example 7, wherein the one or more struts axially extend from a cuff.

Example 9. The tissue modification device of any one of the preceding examples, but particularly Example 8, wherein, when expanded, the one or more struts are substantially parallel to one another, such that adjacent struts are at the substantially equal distance.

Example 10. The tissue modification device of any one of the preceding examples, but particularly Example 5, wherein the scaffold comprises a shape memory material.

Example 11. The tissue modification device of any one of the preceding examples, but particularly Example 1, wherein: the electrode array comprises a plurality of zones; and an RF field generator is configured to supply the electrical current to one or more zones of the plurality of zones for selective ablation of the target tissue.

Example 12. The tissue modification device of any one of the preceding examples, but particularly Example 11, wherein the plurality of zones are angular zones, linear zones, or a combination thereof.

Example 13. The tissue modification device of any one of the preceding examples, but particularly Example 1, wherein the substantially equal distance is between about 2 mm and about 6 mm.

Example 14. The tissue modification device of any one of the preceding examples, but particularly Example 1, wherein the expandable support structure comprises a balloon support.

Example 15. The tissue modification device of any one of the preceding examples, but particularly Example 1, wherein the electrode array is printed directly on the expandable support structure.

Example 16. A system for debulking of a target tissue from a reusable tissue modification device in a vessel, the system comprising: an elongate body comprising a tissue modification device on a distal end of the elongate body, the tissue modification device comprising: a scaffold comprising an electrode array with about evenly spaced adjacent electrodes; an expandable support structure coupled to the scaffold and configured to expand the scaffold; and a radiofrequency (RF) generator configured to supply an electrical current to the electrode array and generate a RF field for modification of the target tissue.

Example 17. The system of any one of the preceding examples, but particularly Example 16, further comprising one or more temperature sensors disposed on the scaffold or the expandable support structure.

Example 18. The system of any one of the preceding examples, but particularly Example 17, wherein the one or more temperature sensors are configured to provide real-time feedback to the RF field generator to adjust power output generated by the RF field.

Example 19. The system of any one of the preceding examples, but particularly Example 16, wherein the scaffold is capable of generating the RF field.

Example 20. The system of any one of the preceding examples, but particularly Example 19, wherein the scaffold comprises a shape memory material that comprises Nitinol.

Example 21. The system of any one of the preceding examples, but particularly Example 16, wherein the scaffold comprises a plurality of struts, wherein the electrode array is carried by at least one strut of the plurality of struts.

Example 22. The system of any one of the preceding examples, but particularly Example 16, wherein: the electrode array comprises one or more zones; and the RF field generator is configured to supply the electrical current to a zone of the one or more zones for selective ablation of the target tissue.

Example 23. The system of any one of the preceding examples, but particularly Example 22, wherein each electrode zone comprises a temperature sensor electrically coupled to a control system using independent conductors.

Example 24. The system of any one of the preceding examples, but particularly Example 16, wherein: the electrode array comprises a plurality of zones; and the RF field generator is configured to supply the electrical current to one or more zones of the plurality of zones for selective ablation of the target tissue.

Example 25. The system of any one of the preceding examples, but particularly Example 24, wherein each electrode zone comprises a temperature sensor electrically coupled to a control system using independent conductors.

Example 26. The system of any one of the preceding examples, but particularly Example 24, wherein the plurality of zones are angular zones, linear zones, or a combination thereof.

Example 27. The system of any one of the preceding examples, but particularly Example 16, wherein the expandable support structure comprises a balloon support.

Example 28. The system of any one of the preceding examples, but particularly Example 16, wherein the expandable support structure is configured to expand to an expansion pressure of at least about 10 atm.

Example 29. The system of any one of the preceding examples, but particularly Example 16, wherein the elongate body defines a lumen configured to receive a fluid therethrough to expand the expandable support structure.

Example 30. The system of any one of the preceding examples, but particularly Example 16, wherein the electrode array comprises bipolar electrodes.

Example 31. The system of any one of the preceding examples, but particularly Example 16, wherein the electrode array comprises monopolar electrodes.

Example 32. The system of any one of the preceding examples, but particularly Example 16, wherein the electrode array comprises monopolar electrodes and bipolar electrodes.

Example 33. The system of any one of the preceding examples, but particularly Example 18, wherein the RF field generator is configured to switch between activation of the electrodes as monopolar RF or bipolar RF.

Example 34. The system of any one of the preceding examples, but particularly Example 16, wherein the RF field generator is configured to change operation modes between bipolar RF and monopolar RF.

Example 35. The system of any one of the preceding examples, but particularly Example 16, further comprising an outer sheath configured to be axially translated over the scaffold and the expandable support structure in an unexpanded configuration.

Example 36. The system of any one of the preceding examples, but particularly Example 16, further comprising a guiding mechanism configured to be axially translatable through a lumen defined at least in part by the scaffold and the expandable support structure.

Example 37. The system of any one of the preceding examples, but particularly Example 36, wherein the guiding mechanism comprises a guidewire.

Example 38. The system of any one of the preceding examples, but particularly Example 35, further comprising a handle removably couplable to an outer sheath of the elongate body, wherein the handle defines: a first channel configured to receive the guiding mechanism therethrough; a second channel configured to receive an inflation fluid therethrough to expand the expandable support structure; and a third channel configured to receive electrical connectors that electrically connect the RF field generator to the electrode array.

Example 39. The system of any one of the preceding examples, but particularly Example 16, wherein the tissue modification device comprises radiopaque markers configured to indicate rotational orientation of the electrode array under fluoroscopy.

Example 40. The system of any one of the preceding examples, but particularly Example 38, wherein the handle comprises indicia corresponding to one or more radiopaque markers disposed on the tissue modification device to provide rotational orientation feedback.

Example 41. The system of any one of the preceding examples, but particularly Example 38, wherein the handle comprises a user interface configured to enable selective activation of one or more zones of the electrode array.

Example 42. The system of any one of the preceding examples, but particularly Example 16, wherein the RF field generator is configured to generate a plurality of RF fields.

Example 43. The system of any one of the preceding examples, but particularly Example 42, wherein the plurality of RF fields comprises three different RF fields.

Example 44. The system of any one of the preceding examples, but particularly Example 16, wherein the RF field generator comprises an impedance sensor.

Example 45. The system of any one of the preceding examples, but particularly Example 44, wherein the impedance sensor is configured to provide feedback to the RF field generator to adjust RF energy output of the electrode array.

Example 46. The system of any one of the preceding examples, but particularly Example 16, further comprising: a control system comprising a processor and a memory; and a temperature sensor disposed on the tissue modification device and communicatively coupled to the control system, wherein the processor is configured to execute instructions stored in the memory, the instructions comprising: activating the RF field generator to apply the electrical current to the electrode array; receiving a signal from the temperature sensor representing a temperature of the target tissue; in response to detecting the signal indicating the temperature is outside of a predefined temperature range, altering the activation of the RF field generator; and in response to detecting the signal indicating the temperature is within the predefined temperature range, maintaining the activation of the RF field generator.

Example 47. The system of any one of the preceding examples, but particularly Example 46, wherein the predefined temperature range is between about 40 degrees Celsius and about 100 degrees Celsius.

Example 48. The system of any one of the preceding examples, but particularly Example 16, further comprising: a control system comprising a processor and a memory; and an impedance sensor disposed on the tissue modification device and communicatively coupled to the control system, wherein the processor is configured to execute instructions stored in the memory, the instructions comprising: activating the RF field generator to supply the electrical current to the electrode array; receiving a signal from the impedance sensor; when the signal indicates that an impedance of the tissue modification device is outside of a predefined impedance range, altering activation of the RF field generator; and when the signal indicates that the impedance of the tissue modification device is within the predefined impedance range, maintaining activation of the RF field generator.

Example 49. The system of any one of the preceding examples, but particularly Example 48, wherein the predefined impedance range is about 30 Ohms to about 600 Ohms.

Example 50. The system of any one of the preceding examples, but particularly Example 16, wherein the scaffold or the expandable support structure comprises one or more radiopaque elements for fluoroscopic visualization.

Example 51. The system of any one of the preceding examples, but particularly Example 16, wherein the expandable support structure is configured to expand to a diameter of about 4 mm to about 20 mm.

Example 52. The system of any one of the preceding examples, but particularly Example 16, further comprising: a control system comprising a processor and a memory and communicatively coupled to the electrode array; and a graphical user interface communicatively coupled to the control system, wherein the processor is configured to execute instructions stored in the memory, the instructions comprising: receiving an input indicating one or more zones of the electrode array for activation; activating the one or more zones; and causing display, on the graphical user interface, one or more of: a temperature, a power, or an impedance of the tissue modification device.

Example 53. The system of any one of the preceding examples, but particularly Example 16, further comprising: a control system comprising a processor and a memory and communicatively coupled to the electrode array, wherein the processor is configured to execute instructions stored in the memory, the instructions comprising: receiving an input indicating one or more zones of the electrode array for activation; activating the one or more zones; and monitoring one or more of: a temperature, a power, or an impedance of the tissue modification device.

Example 54. A method of treating a target lesion, the method comprising: advancing a tissue modification device to a target tissue comprising the target lesion, wherein the tissue modification device comprises: an expandable support structure, and an electrode array at least partially surrounding the expandable support structure, wherein, when the expandable support structure is expanded, the electrode array comprising adjacent electrodes that are evenly spaced; expanding the expandable support structure to expand the electrode array; and generating a plurality of zones of radiofrequency (RF) fields through at least a part of the electrode array to activate the electrode array to ablate the target tissue.

Example 55. The method of any one of the preceding examples, but particularly Example 54, further comprising manipulating a scaffold comprising the electrode array to target the target tissue with another RF field of the RF fields generated from another zone of the plurality of zones.

Example 56. The method of any one of the preceding examples, but particularly Example 55, wherein manipulating comprises rotating the scaffold to align one or more zones of the plurality of zones with the target tissue.

Example 57. The method of any one of the preceding examples, but particularly Example 54, wherein the expandable support structure comprises a balloon.

Example 58. The method of any one of the preceding examples, but particularly Example 54, wherein the electrode array comprises bipolar electrodes, and wherein the method further comprises adjusting one or more of the RF fields to generate a bipolar RF.

Example 59. The method of any one of the preceding examples, but particularly Example 58, further comprising penetrating the target tissue to a depth of about 2 mm to about 6 mm.

Example 60. The method of any one of the preceding examples, but particularly Example 54, wherein the electrode array comprises monopolar electrodes, and wherein the method further comprises adjusting one or more of the RF fields to generate a monopolar RF.

Example 61. The method of any one of the preceding examples, but particularly Example 60, further comprising penetrating the target tissue to a depth of about 4 mm to about 10 mm.

Example 62. The method of any one of the preceding examples, but particularly Example 54, wherein the electrode array comprises monopolar electrodes and bipolar electrodes, and wherein the method further comprises switching between activation of the monopolar electrodes and the bipolar electrodes.

Example 63. The method of any one of the preceding examples, but particularly Example 54, wherein the electrode array is at least partially embedded in a scaffold.

Example 64. The method of any one of the preceding examples, but particularly Example 63, wherein the advancing comprises advancing the scaffold and the expandable support structure to a lumen of an implantable device comprising a restenotic lesion.

Example 65. The method of any one of the preceding examples, but particularly Example 64, wherein the implantable device comprises a stent.

Example 66. The method of any one of the preceding examples, but particularly Example 64, further comprising restoring patency of the lumen of the implantable device.

Example 67. The method of any one of the preceding examples, but particularly Example 54, further comprising axially translating an outer sheath to unsheathe the electrode array and the expandable support structure.

Example 68. The method of any one of the preceding examples, but particularly Example 54, further comprising: contracting the expandable support structure; and removing the expandable support structure from the target tissue.

Example 69. The method of any one of the preceding examples, but particularly Example 54, further comprising: contracting the expandable support structure; re-positioning the tissue modification device; and re-deploying the expandable support structure.

Example 70. The method of any one of the preceding examples, but particularly 69, wherein the contracting, re-positioning, and re-deploying occur more than one time.

Example 71. The method of any one of the preceding examples, but particularly Example 54, further comprising axially translating an outer sheath to cover the electrode array and the expandable support structure.

Example 72. The method of any one of the preceding examples, but particularly Example 54, further comprising deactivating one or more of the RF fields after about 30 seconds to about 200 seconds.

Example 73. The method of any one of the preceding examples, but particularly Example 54, further comprising selectively activating one or more zones of the electrode array to selectively ablate a portion of the target tissue.

Example 74. The method of any one of the preceding examples, but particularly Example 54, wherein the expanding the expandable support structure comprises delivering a fluid through the tissue modification device to expand the expandable support structure.

Example 75. The method of any one of the preceding examples, but particularly Example 54, wherein the expanding the expandable support structure comprises expanding the expandable support structure to an expansion pressure of at least about 4 atm.

Example 76. The method of any one of the preceding examples, but particularly Example 54, further comprising ablating the target tissue at a temperature between about 40 degrees Celsius and about 100 degrees Celsius.

Example 77. A system for treating restenosis of an implantable device, comprising: an elongate body comprising an expandable support structure at least partially surrounded by an electrode array on a distal end of the elongate body, wherein the electrode array is configured for directional ablation, and wherein the expandable support structure is configured to expand the electrode array against a lesion associated with the implantable device; and a control system comprising a radiofrequency (RF) field generator configured to selectively activate angular zones of the electrode array, wherein the control system is configured to perform a feedback loop to maintain an ablation temperature within a therapeutic range.

Example 78. The system of any one of the preceding examples, but particularly Example 77, wherein the electrode array is configured to direct an ablating energy field to generate the ablation temperature.

Example 79. The system of any one of the preceding examples, but particularly Example 78, wherein the control system is configured to modify the activated angular zones of the electrode array to target the lesion with another ablating energy field.

Example 80. The system of any one of the preceding examples, but particularly Example 78, wherein the ablating energy field is configured to be directed to a depth of about 2 mm to about 6 mm.

Example 81. The system of any one of the preceding examples, but particularly Example 78, wherein the ablating energy field is configured to penetrate the lesion to a depth of about 4 mm to about 10 mm.

Example 82. The system of any one of the preceding examples, but particularly Example 77, wherein the electrode array is at least partially embedded in a scaffold.

Example 83. The system of any one of the preceding examples, but particularly Example 82, wherein the scaffold comprises nitinol.

Example 84. The system of any one of the preceding examples, but particularly Example 77, wherein the expandable support structure is configured to contract for removal of the electrode array from the lesion.

Example 85. The system of any one of the preceding examples, but particularly Example 77, wherein the control system is configured to activate the angular zones for about 30 seconds to about 200 seconds.

Example 86. The system of any one of the preceding examples, but particularly Example 77, wherein the control system is configured to selectively activate the angular zones of the electrode array to selectively ablate another lesion associated with the implantable device.

Example 87. The system of any one of the preceding examples, but particularly Example 77, wherein the expandable support structure expands with an expansion pressure of at least about 4 atm.

Example 88. The system of any one of the preceding examples, but particularly Example 77, wherein the therapeutic range is between about 40 degrees Celsius and about 100 degrees Celsius.

Example 89. The system of any one of the preceding examples, but particularly Example 77, further comprising a handle removably couplable to the elongate body, wherein the handle defines: a first channel configured to receive a guiding mechanism therethrough; a second channel configured to receive an inflation fluid therethrough to expand the expandable support structure; and a third channel configured to receive electrical connectors that electrically connect the RF field generator to the electrode array.

Example 90. The system of any one of the preceding examples, but particularly Example 89, wherein the expandable support structure is configured to expand by delivering the inflation fluid through the second channel to the expandable support structure.

Example 91. A system for debulking of a target tissue from a reusable tissue modification device in a vessel, the system comprising: an elongate body comprising a tissue modification device on a distal end of the elongate body, the tissue modification device comprising: an electrode array coupled to a scaffold, wherein: the scaffold configured to expand and contract, and when expanded, the scaffold is configured to space adjacent electrodes of the electrode array at a substantially equal distance; and a radiofrequency (RF) field generator configured to supply an electrical current to the electrode array and generate an RF field for modification of the target tissue.

Example 92. The system of any one of the preceding examples, but particularly Example 91, further comprising one or more temperature sensors disposed on the scaffold.

Example 93. The system of any one of the preceding examples, but particularly Example 92, wherein the one or more temperature sensors are configured to provide real-time feedback to the RF field generator to adjust power output generated by the RF field.

Example 94. The system of any one of the preceding examples, but particularly Example 91, wherein the scaffold comprises a shape memory material that comprises Nitinol.

Example 95. The system of any one of the preceding examples, but particularly Example 91, wherein the scaffold comprises a plurality of struts, wherein the electrode array is carried by at least one strut of the plurality of struts.

Example 96. The system of any one of the preceding examples, but particularly Example 91, wherein: the electrode array comprises one or more zones; and the RF field generator is configured to supply the electrical current to a zone of the one or more zones for selective ablation of the target tissue.

Example 97. The system of any one of the preceding examples, but particularly Example 91, wherein: the electrode array comprises a plurality of zones; and the RF field generator is configured to supply the electrical current to one or more zones of the plurality of zones for selective ablation of the target tissue.

Example 98. The system of any one of the preceding examples, but particularly Example 97, wherein the plurality of zones are angular zones, linear zones, or a combination thereof.

Example 99. The system of any one of the preceding examples, but particularly Example 91, wherein the scaffold is radially expandable from a cuff.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “energy field” may include, and is contemplated to include, a plurality of energy fields. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A tissue modification device for modifying a target tissue within a vascular stent, the tissue modification device comprising:
an elongate body;
a tissue modification structure on a distal end of the elongate body, the tissue modification structure comprising:
an expandable support; and
an electrode array carried by the expandable support, the electrode array having at least a first and a second electrically isolated zones positioned circumferentially around the expandable support;
wherein, when expanded, the expandable support is configured to advance the electrode array into contact with the target tissue within the vascular stent; and
wherein the tissue modification device is configured to deliver at least a first tissue modifying energy field from the first electrically isolated zone when a first electrical current is supplied to the electrode array, and optionally to deliver a second tissue modifying energy field from the second electrically isolated zone when a second electrical current is supplied to the electrode array.

2. The tissue modification device of claim 1, further comprising an electrically conductive cuff carried by the elongate body.

3. The tissue modification device of claim 2, further comprising a plurality of insulated, electrically conductive ramps extending distally from the electrically conductive cuff.

4. The tissue modification device of claim 3 wherein the electrically conductive ramps place the electrically conductive cuff into electrical communication with the electrode array.

5. The tissue modification device of claim 4, wherein the electrode array comprises a plurality of electrodes carried by a plurality of axially extending struts extending distally from the electrically conductive ramps.

6. The tissue modification device of claim 5, wherein the first electrically isolated zone comprises a first plurality of axially extending struts, and the second electrically isolated zone comprises a second plurality of axially extending struts.

7. The tissue modification device of claim 6, wherein one or both of the first and second plurality of struts comprise Nitinol.

8. The tissue modification device of claim 5, wherein the expandable support is expandable from a first, reduced cross section for translumenal navigation and a second, expanded cross section for positioning the electrode array adjacent to target tissue within the stent.

9. The tissue modification device of claim 8, wherein adjacent axially extending struts are spaced apart within a range of from about 2 mm to about 6 mm when the expandable support is in the second expanded cross section.

10. The tissue modification device of claim 1, wherein the expandable support comprises a vascular occlusion balloon.

11. The tissue modification device of claim 10, wherein the vascular occlusion balloon is configured to expand to an expansion pressure of at least about 10 atm.

12. The tissue modification device of claim 2, wherein the electrically conductive cuff comprises an indicium of rotational orientation.

13. The tissue modification device of claim 12, wherein the indicium comprises a fluoroscopically visible aperture extending through the electrically conductive cuff.

14. The tissue modification device of claim 13, wherein the fluoroscopically visible aperture points in a lateral direction relative to a longitudinal axis of the elongate body.

15. The tissue modification device of claim 1, wherein the electrode array is bonded to the expandable support.

16. The tissue modification device of claim 1, wherein the electrode array is printed directly on the expandable support.

17. The tissue modification device of claim 1, comprising at least one temperature sensor carried by the tissue modification structure.

18. The tissue modification device of claim 1, further comprising a proximal handle which includes:
a first channel configured to receive a guidewire therethrough;
a second channel configured to receive an inflation fluid therethrough to expand the expandable support; and
a third channel configured to receive electrical conductors configured to electrically connect an RF field generator to the electrode array.
